(19) United States
(12) Patent Application Publication
(10) Pub. No.: US 2021/0071138 A1
(43) Pub. Date: Mar. 11, 2021

(54) GAMETOGENESIS

(71) Applicant: United Kingdom Research and Innovation, Swindon, Wiltshire (GB)

(72) Inventors: Petra HAJKOVA, Swindon, Wiltshire (GB); Peter HILL, Swindon, Wiltshire (GB); Harry LEITCH, Swindon, Wiltshire (GB)

(73) Assignee: United Kingdom Research and Innovation, Swindon, Wiltshire (GB)

(21) Appl. No.: 16/955,716

(22) PCT Filed: Dec. 21, 2018
(86) PCT No.: PCT/EP2018/086642

§ 371 Date: Jun. 18, 2020

(30) Foreign Application Priority Data
Dec. 22, 2017 (GB) 1721724.1

(51) Int. Cl. C12N 5/0735; C12N 5/075; C12N 5/076

(52) U.S. Cl. CPC C12N 5/0611; C12N 5/0609; C12N 5/061; C12N 2506/04; C12N 2501/65; C12N 2510/00; C12N 2506/45; C12N 2501/999

(57) ABSTRACT

The present invention relates to in vitro methods of inducing gametogenesis by producing meiotically competent cells. Reagents and kits for use in the methods of the invention are also provided. The present invention finds use in the field of medicine, particularly in the study and treatment of infertility.

### FIELD OF THE INVENTION

The present invention relates to methods of inducing gametogenesis in vitro. Reagents and kits for use in the methods of the invention are also provided. The present invention finds use in the field of medicine, particularly in the study and treatment of infertility.

### BACKGROUND

Gametogenesis is the process by which gametes are generated. In animals, gametogenesis proceeds via the division and differentiation of meiotically competent gonocytes in the gonads (testes in males; ovaries in females). In males, spermatogenesis occurs in the testes to produce spermatozoa from spermatogonial stem cells (SSCs) in a multi-step process involving both meiosis and mitosis. The SSCs arise from gonocytes in the postnatal testes and these gonocytes, in turn, arise from primordial germ cells (PGCs; Phillips et al, 2010), which migrate to the genital ridge during embryogenesis.

Specification of PGCs (embryonic precursors of gonocytes) begins around embryonic day 6.25 (E 6.25) in mice¹. Following specification, the nascent PGCs undergo pronounced global epigenetic changes²⁻⁹, including global reduction of genomic 5-methylcytosine (5mC)^{3,6,7,10}. Following their migration through the developing embryo, further epigenetic reprogramming, which includes global DNA demethylation, proceeds once the PGCs arrive to the developing embryonic gonad. Molecular mechanism(s) implicated in this DNA demethylation of gonadal PGCs have been the focus of intense studies^{3,4,6,12-19,21} and recently published observations suggest that the 5mC oxygenase Tet1 is a critical factor involved in the correct progression of DNA demethylation in gonadal PGCs^{12,14,16,17}. However, the precise nature of this epigenetic reprogramming has remained elusive. Recent work has shown (Hill et al, 2018) that gonadal epigenetic reprogramming is critically involved in the PGC-to-gonocyte transition, which is required to produce meiotically competent gonocytes (and thus allow gametogenesis to be initiated). Importantly, the Gonadal reprogramming process represents a barrier that until recently has only been overcome in the context of the gonadal somatic environment^{5,24,25,27}.

Recent studies have reported the conversion of somatic precursor cells to meiotically competent cells via induced expression of several germ line-related genes (Medrano et al, 2016). Other studies have identified Tet1 as the critical factor in regulating certain germ line-related genes during the activation of female gametogenesis¹⁶. However, manipulation of Tet1 expression has not been shown to be sufficient to convert somatic precursor cells to meiotically competent cells.

In humans, infertility is a major health problem. For instance, male infertility affects 7% of the population, with around 10% of infertile men being azoospermic (Galdon et al, 2016). The provision of meiotically competent cells represents an important step in the in vitro recapitulation of gametogenesis, which will find utility in research and medicine, particularly in the context of infertility.

### SUMMARY OF THE INVENTION

The inventors have found that two distinct biochemical conditions are required for effective activation of a set of genes required for the progression from PGC to gonocyte stage of germline development (the genes are termed the “germline reprogramming-responsive genes (GRR genes)” herein, and in Hill et al 2018). These genes, required also for the conversion of somatic precursor cells, pluripotent cells or early germ cells into meiotically competent cells, can be activated through firstly a reduction of DNA methylation, and secondly the removal of polycomb driven repression. Once these biochemical conditions are in place, transcriptional factors and activators including the epigenetic activator Tet1 are able to drive GRR gene expression. The recruitment of transcriptional activators such as Tet1 and/or the expression of GRR genes are indicative of the conversion of the precursor (somatic) cell into a meiotically competent cell.

Accordingly, in a first aspect, the invention provides an in vitro method of producing a meiotically competent cell, the method comprising:
(i) providing a precursor cell,
(ii) inhibiting methylation of the genomic DNA of the precursor cell,
(iii) treating the precursor cell with an inhibitor of a polycomb repressive complex, and then
(iv) propagating the precursor cell for a period of time and under culture conditions suitable for the precursor cell to become a meiotically competent cell;
wherein step (ii) and step (iii) may be performed simultaneously or sequentially in either order.

In some embodiments, the precursor cell is derived from a sample that has been obtained from a subject. The precursor cell may be a stem cell, a primordial germ cell-like cell (PGCLC) or, an early germ cell. In some embodiments, the stem cell is an induced pluripotent stem cell (iPS cell) or a spermatogonial stem cell In some embodiments, the inhibiting step (ii) and the treating step (iii) result in the induction of expression of germline reprogramming responsive (GRR) genes by the precursor cell during propagating step (iv). The expression of the GRR genes may be associated with or induced by recruitment of a transcriptional activator, for instance Tet1. Tet1 may be expressed by the precursor cell, and/or Tet1 may be exogenously provided (e.g. by delivering a nucleic acid that exogenously expresses Tet1, by enhancing or stimulating the endogenous expression of Tet1 and/or by providing Tet1 in the form of an exogenous protein).

Exogenously provided Tet1 may be in the form of a fusion construct that is targeted to one or more specific genomic regions. For instance, a Tet1 fusion construct may be targeted to promoter or enhancer sequences involved in expressing one or more of the GRR genes disclosed herein. Providing an effective level of Tet1 as a transcriptional activator enhances the expression of GRR genes. The methods of this invention enable GRR gene expression to be enhanced, and these methods may include increasing or inducing Tet1 expression and/or targeting Tet1 to one or more GRR genes.

The method of the invention may also include the detection and/or quantification of the expression level of one or more GRR genes in the cell. The GRR genes are listed in Table 1. Methods for detecting and/or quantifying expression levels are well known in the art. For instance, mRNA levels of the gene can be measured e.g. by RT-PCR. Protein expression levels can be measured e.g. by assays such as ELISA. Expression of the one or more GRR genes can be measured before, during or after the conversion of the precursor cell to the meiotically competent cell. Preferably, the expression of one or more GRR genes is measured in the meiotically competent cell following step (iv). The GRR gene to be measured may be one or more of Dazl, Hormad1, Sycp2, Sycp3, Mae1, Fkbp6 (see Table 1). In some embodiments of this invention, the inhibitor of polycomb repressive complex is a PRC1 inhibitor (meaning that the PRC1 complex is selectively inhibited. In other embodiments of this invention, the inhibitor of polycomb repressive complex is a PRC2 inhibitor (meaning that the PRC2 complex is selectively inhibited). In yet further embodiments, the inhibitor of polycomb repressive complex inhibits both PRC1 and PRC2.

In some embodiments, the inhibitor is of polycomb repressive complex is PRT4165. In other embodiments, the inhibitor of polycomb repressive complex is an RNAi molecule, which selectively knocks-down the expression of a component of a polycomb repressive complex, e.g. a component of PRC1 or PRC2.

In some embodiments of the invention, the inhibition of DNA methylation (step (ii) of the method) is performed by treating the precursor cell with an agent that reduces genomic DNA methylation. In the context of this disclosure, ‘treating’ the cell is understood to mean ‘contacting’ the cell, i.e. exposing the cell to an agent. Furthermore, ‘inhibiting’ includes both ‘reducing’ and ‘completely preventing’. For instance, the precursor cell may be treated (contacted) with a DNA methyltransferase inhibitor, with an agent that prevents the deposition of DNA methylation, or with an agent that inhibits the maintenance of DNA methylation. 5-aza-2-deoxycytidine (5-Aza-dc) is an agent that inhibits DNA methylation and also inhibits the maintenance of DNA methylation.

In embodiments where the agent that reduces genomic DNA methylation is a DNA methyltransferase inhibitor, the DNA methyltransferase inhibitor may be a DNMT1 inhibitor. For instance, the DNA methyltransferase inhibitor may be SGI 1027 or 5-azacytidine. Alternatively, the DNA methyltransferase inhibitor may be an RNAi molecule, which knocks-down expression of a component of the DNA methylation machinery. The RNAi molecule may be an siRNA molecule or an miRNA molecule (or a precursor of either).

In alternative embodiments, the inhibition of DNA methylation (step (ii) of the method) may be performed by using a technique such as gene-editing to inactivate a DNA methyltransferase gene. Accordingly, various means of inhibiting DNA methylation can be used in producing the meiotically competent cell. For instance, genetic knock-out of the methylation machinery or chemical blockade of the methylation machinery can be used.

In a second aspect, this invention provides a meiotically competent cell produced by the methods described herein. The meiotically competent cell may be treated with retinoic acid. Retinoic acid is known to induce gametogenesis in meiotically competent cells.

Accordingly, in a third aspect, this invention provides a method of inducing gametogenesis in a meiotically competent cell of the invention, by treating it with retinoic acid. In some embodiments, the gametogenesis is spermatogenesis. In other embodiments, the gametogenesis is oogenesis.

In a further aspect, this invention provides a kit for the in vitro production of meiotically competent cells. The kit of the invention comprises a methylation inhibitor and an inhibitor of a polycomb repressive complex. In some embodiments, the kit also comprises retinoic acid. The kit may also comprise appropriate hardware to be used in the methods of the invention, e.g. test tubes, culture plates, etc.

In a yet further aspect, this invention provides a method of assessing the fertility of a mammal. In this aspect of the invention, the nucleic acid sequence and/or epigenetic status and/or gene expression level of one or more germline reprogramming responsive (GRR) genes is determined in a cell that has been obtained from the mammal.

In a related aspect, this invention provides a method of determining the meiotic competency of a cell, the method comprising determining the nucleic acid sequence, epigenetic status, and/or expression of one or more germline reprogramming responsive (GRR) genes in the genomic DNA of the cell. This invention also provides kits and/or assay plates having a group of probes, which group consists of, or consists essentially of, probes that detect expression or epigenetic status or expression of one or more GRR genes as set forth in Table 1.

TABLE 1
GRR genes

| | Effect of genetic | |
|---|---|---|
| | knockout on germ | Mechanism of |
| Gene ID | cell development | transcriptional activation |
| 1700013H16Rik | n.d. | 5mC reprogramming driven |
| 1700018B24Rik | n.d. | 5mC reprogramming driven |
| 1700029P11Rik | n.d. | 5mC reprogramming driven |
| 4921515J06Rik | n.d. | Reprogramming |
| | | independent/insufficient |
| 4930467D21Rik | n.d. | l.c.c. |
| 4933416C03Rik | n.d. | l.c.c. |
| 8030474K03Rik | n.d. | Reprogramming |
| | | independent/insufficient |
| Adad1 | Severe germ cell | l.c.c. |
| | defect (in males)⁵⁴ | |
| Asz1 | Severe germ cell | 5mC reprogramming driven |
| | defect (in males)⁵⁵ | |
| AU022751 | n.d. | 5mC reprogramming driven |
| Brdt | Severe germ cell | Reprogramming |
| | defect (in males)⁵⁶ | independent/insufficient |
| D1Pas1 | n.d. | 5mC reprogramming driven |
| Dazl | Severe germ cell | 5mC/PRC1 |
| | defect⁵⁷ | reprogramming driven |
| Ddx4 | Severe germ cell | Reprogramming |
| | defect (in males)⁵⁸ | independent/insufficient |
| Dpep3 | n.d. | 5mC reprogramming driven |
| Fam178b | n.d. | l.c.c. |
| Fkbp6 | Severe germ cell | 5mC reprogramming driven |
| | defect (in males)⁵⁹ | |
| Gm13718 | n.d. | l.c.c. |
| Gm16270 | n.d. | l.c.c. |
| Gm2382 | n.d. | Reprogramming |
| | | independent/insufficient |
| Gm7061 | n.d. | 5mC/PRC1 |
| | | reprogramming driven |
| Hormad1 | Severe germ | 5mC reprogramming driven |
| | cell defect⁶⁰ | |
| Hsf2bp | n.d. | 5mC reprogramming driven |
| Hsf5 | n.d. | l.c.c. |
| Ly6k | Severe germ cell | l.c.c. |
| | defect (in males)⁶¹ | |
| Mael | Severe germ cell | PRC1 reprogramming driven |
| | defect (in males)⁶² | |
| Mov10l1 | Severe germ cell | 5mC reprogramming driven |
| | defect (in males)⁶³ | |
| Naa11 | n.d. | 5mC reprogramming driven |
| Phf17 | n.d. | PRC1 reprogramming driven |
| Pnldc1 | n.d. | PRC1 reprogramming driven |
| Rad51c | Severe germ | l.c.c. |
| | cell defect⁶⁴ | |
| Rhox13 | n.d. | 5mC reprogramming driven |
| Rpl10l | n.d. | 5mC reprogramming driven |
| Sec1 | n.d. | l.c.c. |
| Slc25a31 | n.d. | l.c.c. |
| Stk31 | Dispensable for | 5mC reprogramming driven |
| | reproduction⁶⁵ | |
| Sycp1 | Severe germ | 5mC reprogramming driven |
| | cell defect⁶⁶ | |
| Sycp2 | Severe germ | 5mC/PRC1 |
| | cell defect⁶⁷ | reprogramming driven |
| Sycp3 | Severe germ | PRC1 reprogramming driven |
| | cell defect⁶⁸ | |
| Taf7l | Severe germ cell | 5mC/PRC1 |
| | defect (in males)⁶⁹ | reprogramming driven |
| Taf9b | n.d. | 5mC reprogramming driven |
| Tdrd1 | Severe germ cell | Reprogramming |
| | defect (in males)⁷⁰ | independent/insufficient |
| Tex12 | n.d. | 5mC/PRC1 |
| | | reprogramming driven |
| Tex15 | Severe germ cell | 5mC/PRC1 |
| | defect (in males)⁷¹ | reprogramming driven |
| Trim52 | n.d. | 5mC reprogramming driven |

n.d.: no data;
l.c.c.: low confidence classification

As described herein, some embodiments of this invention involve detecting GRR genes (e.g. sequence, epigenetic status or expression level) or inducing GRR expression. These embodiments may involve the detection or induction of a group of genes comprising, consisting of, or consisting essentially of, one or more GRR genes selected from Table 1; for instance any 2, any 3, any 4, any 5, any 6, any 7, any 8, any 9, any 10, any 11, any 12, any 13, any 14, any 15, any 16, any 17, any 18, any 19, or any 20 genes selected from Table 1. In some embodiments, the genes selected from Table 1 may include one or more of Dazl, Hormad1, Sycp2, Sycp3, Mae1, Fkbp6. In other embodiments, the genes selected from Table 1 may exclude any or all of Dazl, Hormad1, Sycp2, Sycp3, Mae1, Fkbp6.

Therapeutic Applications

The methods and products of this invention have therapeutic application, particularly in the treatment of infertility. For instance, as described herein, the meiotically competent cell produced by the methods of the invention can be induced to undergo gametogenesis, e.g. by treatment with retinoic acid (RA). Gametocytes produced in this way (i.e. spermatocytes; oocytes) constitute further aspects of this invention. The gametocyte of the invention has therapeutic applications, for instance in the adoptive transfer to an infertile individual: It is envisaged that the spermatocyte of the invention may be adoptively transferred to the testes of a male infertility patient. It is envisaged that the oocyte of the invention may be adoptively transferred to the ovary of a female infertility patient. These gametocytes may be derived from the patient's own cells, e.g. by performing the method of the invention on an iPS cell, a spermatogonial stem cell (SSC), or a PGCLC derived from a patient cell. This approach allows autologous adoptive transfer of the gametocyte to the patient.

In further aspects of the invention, gametes are derived from the abovementioned gametocyte of the invention in vitro. In this way, the invention provides male gametes, spermatozoa (sperm), and female gametes, ova (eggs), which may be used therapeutically. For instance, the gametes of the invention may be used in in vitro fertilisation (IVF) applications.

Precursor Cells

As explained herein, the methods of the invention are capable of converting a somatic precursor cell into a meiotically competent cell. This subsection discusses various types of cell that may be used as the precursor cell.

In nature, the precursor to meiotically competent gonocytes are primordial germ cells. Current in vitro systems aimed at generating PGC-like cells (PGCLCs)^{5,24-26} can successfully recapitulate only the early stages of PGC development, with gonadal reprogramming still presenting a barrier that can be overcome and executed only in the context of the gonadal somatic environment^{5,24,25,27}. In some embodiments of this invention, the precursor cell is a PCGLC obtained by the aforementioned prior art methods.

In other embodiments of this invention, the precursor cell is a stem cell, for instance an embryonic stem cell. Human embryonic stem cells represent one type of precursor cell. It is known in the art that human embryonic stem cells can be obtained without destroying the human embryo (Chung et al., 2008). Mouse embryonic stem cells also represent a type of precursor cell that usefully demonstrate the efficacy of this invention. The inventors have found that the epigenetic regulation of GRR genes in PGCs is very similar to that in serum-grown mouse embryonic stem cells.

Pluripotent stem cells that are not of embryonic origin may also be used as the precursor cells in the methods of this invention. Pluripotent stem cells can be obtained by methods including:

Reprogramming by nuclear transfer. This technique involves the transfer of a nucleus from a somatic cell into an oocyte or zygote. In some situations, this may lead to the creation of an animal-human hybrid cell. For example, cells may be created by the fusion of a human somatic cell with an animal oocyte or zygote or fusion of a human oocyte or zygote with an animal somatic cell.

Reprogramming by fusion with embryonic stem cells. This technique involves the fusion of a somatic cell with an embryonic stem cell. This technique may also lead to the creation of animal-human hybrid cells, as in 1 above.

Spontaneous re-programming by culture. This technique involves the generation of pluripotent cells from non-pluripotent cells after long term culture. For example, pluripotent embryonic germ (EG) cells have been generated by long-term culture of primordial germ cells (PGC) (Matsui et al., 1992). The development of pluripotent stem cells after prolonged culture of bone marrow-derived cells has also been reported (Jiang et al., 2002). They designated these cells multipotent adult progenitor cells (MAPCs). Shinohara et al also demonstrated that pluripotent stem cells can be generated during the course of culture of germline stem (GS) cells from neonate mouse testes, which they designated multipotent germline stem (mGS) cells (Kanatsu-Shinohara et al., 2004).

Reprogramming by definedfactors. For example the generation of iPS cells by the retrovirus-mediated introduction of transcription factors (such as Oct-3/4, Sox2, c-Myc, and KLF4) into mouse embryonic or adult fibroblasts, e.g. as described by Kaji et al., 2002 also describe the non-viral transfection of a single multiprotein expression vector, which comprises the coding sequences of c-Myc, Klf4, Oct4 and Sox2 linked with 2A peptides, that can reprogram both mouse and human fibroblasts. iPS cells produced with this non-viral vector show robust expression of pluripotency markers, indicating a reprogrammed state confirmed functionally by in vitro differentiation assays and formation of adult chimaeric mice. They succeeded in establishing reprogrammed human cell lines from embryonic fibroblasts with robust expression of pluripotency markers. Induced pluripotent stem cells have the advantage that they can be obtained by a method that does not cause the destruction of an embryo, more particularly by a method that does not cause the destruction of a human or mammalian embryo.

Pluripotent stem cells may also be obtained from arrested embryos which stopped cleavage and failed to develop to morula and blastocysts in vitro, obtained by parthenogenesis, or derived from hESC lines from single blastomeres or biopsied blastomeres.

As such, aspects of the invention may be performed or put into practice by using cells that have not been prepared exclusively by a method which necessarily involves the destruction of human or animal embryos from which those cells may be derived. This optional limitation is specifically intended to take account of Decision G0002/06 of 25 Nov. 2008 of the Enlarged Board of Appeal of the European Patent Office.

In other embodiments, gametogonia (gamete stem cells) may be used as the precursor cell. For instance spermatogonial stem cells (SSCs) are one preferred precursor cell type for use in the methods of this invention. SSCs may be extracted from the testes, e.g. from a testes biopsy. Testes aspirate is one source of a cell preparation (extract) that contains SSCs. It is envisaged that the methods of the invention can be performed directly on such testes extracts, or could be performed upon SSCs that have been enriched, selected and/or purified.

The precursor cell may be obtained from a subject. The subject may be a mammalian subject, for instance a human subject. In some embodiments of the invention, the subject is an infertility patient.

RNA Interference (RNAi)

The present invention also includes the use of techniques known in the art for the therapeutic down regulation of a component of the polycomb repressive complex or of a component of DNA methylation machinery. These include the use RNA interference (RNAi).

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNAs are derived by processing of long double stranded RNAs. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of these sequences for down-regulating the expression of components of a polycomb repressive complex, e.g. PRC1 and/or PRC2.

The siRNA is typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA are typically single stranded and have regions that are partially complementary enabling the miRNA to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is known in the art.

### BRIEF DESCRIPTION OF THE DRAWINGS

Embodiments of the invention will now be described by way of example with reference to the accompanying drawings:

FIG. 1—5mC and 5hmC dynamics during epigenetic reprogramming. a) Key events during mouse PGC development. b-c) Individual 5mC (b, left) and 5hmC (b, right) and combined 5mC/5hmC (c) levels in mESCs and E9.5 to E13.5 PGCs (LC/MS). Asterisks in (b) refer to mean values. Adjusted p-values are based on ANOVA and Tukey posthoc test. Bar chart in (c) depicts median value of biological replicates depicted in (b). d) Re-distribution of 5hmC from the uniquely mapped part of the genome to repetitive elements between E10.5 and E12.5. p-values based on combined ANOVA and Tukey post hoc test. e) Representative 5hmC immunostaining in E10.5 and E12.5 PGCs. Scale bar represents 10 μm. Details regarding sample sizes and how samples were collected can be found in Statistics and Reproducibility section.

FIG. 2—Tet1 safeguards but does not drive DNA demethylation. a-b) Representative immunostaining against 5hmC (a) or 5mC (b) in E13.5 wild type and Tet1-KO PGCs. Scale bar represents 10 μm. c-d) Global 5hmC (c) and 5mC (d) levels (LC/MS) in wild type and Tet1-KO PGCs. Sample numbers are indicated on graphs. Asterisks refer to mean values. p-values are based on two-sided Student's t-test. e) Top Figure: Proportion of differentially methylated regions in E14.5 Tet1-KO PGCs (p<0.05, >10% methylation difference; p-value derived from RnBeads software). Bottom Figure: Combined 5mC/5hmC levels (RRBS) in E12.5 (middle) and E14.5 (bottom) Tet1-KO (red) and wild type (blue) PGCs for all E14.5 hypermethylated 2 kB windows. DNA modification levels from E10.5 wild type PGCs are also shown (top panel). Median combined 5mC/5hmC levels are denoted by vertical lines. Details regarding sample sizes and how samples were collected can be found in Statistics and Reproducibility section.

FIG. 3—Germline reprogramming responsive (GRR) genes. a) Combined promoter 5mC/5hmC levels (right), promoter 5hmC levels (centre), or gene expression levels (right) in consecutive stages of PGC development for HCP gene clusters (see Methods). The upper and lower hinges correspond to the first and third quartiles, the middle line corresponds to the median, and the maxima and minima respectively correspond to the highest or lowest value within 1.5× the inter-quartile range. b) Genomic sequences centred on TSSs of methylated and demethylating HCPs (cluster 3, FIG. 3A) ranked based on the significance of up-regulation between E10.5 and E14.5 in wild type PGCs. Each horizontal line represents one gene; the intensity of red indicates the relative enrichment for the feature shown at the top of each column. The TSS+/−5 kb is shown. c) Gene ontology (GO) terms associated with germline reprogramming responsive (GRR) genes; adj. p-value is based on DAVID software. Details regarding sample sizes and how samples were collected can be found in Statistics and Reproducibility section.

FIG. 4—Epigenetic principles of GRR gene activation. a) GRR gene expression dynamics in Tet1-KO PGCs; p-values are based on a two-sided paired Wilcoxon test. b) Combined 5mC/5hmC levels (RRBS) at GRR genes in E12.5 or E14.5 Tet1-KO (red) and wild type (blue) PGCs. For comparison, combined 5mC/5hmC levels in mESCs³⁰ (%; WGBS) are shown. p-values are based on paired two-sided Wilcoxon test. c-d) Log 2-fold change between Dnmt-TKO (green) or Tet1-KO Dnmt-TKO and wild type mESCs (in c) or between wild type+6h PRT4165 treatment (purple), Dnmt-TKO+6h DMSO treatment (green) or Dnmt-TKO+6h PRT4165 treatment (yellow) and wild type+6h DMSO treatment mESCs (in d) for GRR genes and other relevant genes sets. FWER-adjusted p-values are based on GSEA software (see Methods for details). Specific details regarding sample sizes and how samples were collected are found in Statistics and Reproducibility section. For all boxplots, the upper and lower hinges correspond to the first and third quartiles, the middle line corresponds to the median, and the maxima and minima respectively correspond to the highest or lowest value within 1.5× the inter-quartile range.

FIG. 5—Characterisation of WGBS datasets and validation of AbaSeq method. a) Distribution of WGBS coverage for each symmetric CpG. For boxplots, the upper and lower hinges correspond to the first and third quartiles, the middle line corresponds to the median, and the maxima and minima respectively correspond to the highest or lowest value within 1.5× the inter-quartile range. b) Overview of AbaSeq method¹⁵. c-e) Density heatmap showing correlation between 5hmC levels at all 2 kB windows (minimum 4 symmetric CpGs) in E14 mESCs as computed by: (c) TAB-Seq³⁵ (x-axis) and AbaSeq¹⁵ (y-axis); (d) TAB-Seq³⁵ (x-axis) and hMeDIP³⁶ (y-axis); or (e) AbaSeq¹⁵ (x-axis) and hMeDIP³⁶ (y-axis). For (c-e), the Pearson correlation coefficient (p) is shown. Specific details regarding sample sizes and how samples were collected are found in Statistics and Reproducibility section.

FIG. 6—Further analysis of 5hmC levels in E10.5 PGCs. a) Density heatmap showing 5hmC levels per 2 kB window (with minimum 4 CpGs) for E10.5 PGCs (y-axis) and E14 mESCs¹⁵ (y-axis). Pearson correlation coefficient (p) is shown. b) 5hmC levels (AbaSeq) at various regulatory elements in E10.5 PGCs (left) or E14 mESCs¹⁵. p-values based on ANOVA and Dunnett post hoc test. For boxplots, the upper and lower hinges correspond to the first and third quartiles, the middle line corresponds to the median, and the maxima and minima respectively correspond to the highest or lowest value within 1.5× the inter-quartile range. c) Metagene plot showing 5hmC levels (top panel, AbaSeq) and combined 5mC/5hmC levels (bottom panel, WGBS) in E10.5 PGCs across genes expressed at different levels in E10.5 PGCs. d-e) Metagene plot showing 5hmC levels (top panel, AbaSeq) and combined 5mC/5hmC levels (bottom panel, WGBS) in E10.5 PGCs across either CpG islands (d) or across putative active enhancers (e). f) Bar chart showing 5hmC levels at ICRs in E14 mESCs as determined by TAB-Seq³⁵ (%; light green) or AbaSeq¹⁵ (read counts; dark green), or in E10.5 PGCs as determined by AbaSeq (read counts; orange). Specific details regarding sample sizes and how samples were collected are found in Statistics and Reproducibility section.

FIG. 7—Further analysis of 5mC and 5hmC dynamics in PGCs. a) Combined 5mC/5hmC (WGBS; left) or 5hmC (AbaSeq; right) levels at various features within the uniquely mapped part of the genome in PGCs between E10.5 and E12.5. The upper and lower hinges correspond to the first and third quartiles, the middle line corresponds to the median, and the maxima and minima respectively correspond to the highest or lowest value within 1.5× the inter-quartile range. b) The combined 5mC/5hmC (WGBS; left) or 5hmC (AbaSeq; right) levels at various consensus repetitive elements in PGCs between E10.5 and E12.5. Asterisks refer to mean values. For specific details regarding sample sizes and how samples were collected, see the Statistics and Reproducibility section.

FIG. 8—5hmC is targeted to newly hypo-methylated regions following DNA demethylation in mouse gonadal PGCs (see also FIG. 9a) Density heatmap showing Pearson correlation (p) between 5hmC levels for E10.5 biological replicates (left), for E10.5 and E11.5 PGCs (middle), and for E10.5 and E12.5 PGCs (right). b) Mean Z-scores depicting 5hmC (orange, AbaSeq) and combined 5mC/5hmC (grey, WGBS) levels for each stage normalised to the average level of either 5hmC (orange, AbaSeq) or combined 5mC/5hmC (grey, WGBS) across stages. Standard error of the mean is shown by too small to see. c-f) Density heatmap showing the correlation between the total (c,d; y-axis: AbaSeq read counts) or relative (e,f; y-axis: ratio of (AbaSeq read counts)/(%; WGBS)) 5hmC levels in E10.5 (c,e) or E11.5 (d,f) PGCs and the change in combined 5mC/5hmC levels in PGCs between these two stages (x-axis: %; WGBS) for all 2 kB windows with a minimum 20% combined 5mC/5hmC in E10.5 PGCs. g) Density heatmap showing the correlation between the relative 5hmC levels in E11.5 PGCs (y-axis: ratio of (AbaSeq read counts)/(%; WGBS)) and the combined 5mC/5hmC level in E11.5 PGCs (x-axis: %; WGBS) for all 2 kB windows with a minimum 20% combined 5mC/5hmC in E10.5 PGCs. h) Density plot showing the decrease in combined 5mC/5hmC levels in PGCs between E10.5 and E11.5 for 2 kB windows with a minimum 20% total DNA modification in E10.5 PGCs that are either 1) enriched for total 5hmC levels at either E10.5 or E11.5 (green, upper-tail adj. Poisson p-value<0.05), or 2) depleted of total 5hmC at both E10.5 and E11.5 (red, lower-tail adj. Poisson p-value<0.05). i) Combined 5mC/5hmC levels in E10.5 and E11.5 PGCs for 2 kB windows with a minimum 20% combined 5mC/5hmC in E10.5 PGCs that are either 1) enriched for total 5hmC levels at either E10.5 or E11.5 (green, upper-tail adj. Poisson p-value<0.05), or 2) depleted of total 5hmC at both E10.5 and E11.5 (red, lower-tail adj. Poisson p-value<0.05). For all boxplots, the upper and lower hinges correspond to the first and third quartiles, the middle line corresponds to the median, and the maxima and minima respectively correspond to the highest or lowest value within 1.5× the inter-quartile range. p-values are based on a two-sided Wilcoxon test. Note that for density heatmaps: 1) the Spearman correlation (ρₛ) is shown; and 2) the red line represents the smoothed mean as determined by a generalized additive model. Specific details regarding sample sizes and how samples were collected are found in Statistics and Reproducibility section.

FIG. 9—Suggested models implicating 5mC oxidation in DNA demethylation of gonadal PGCs. a) A model of oxidation followed by passive dilution predicts a positive correlation between the extent to which the combined 5mC/5hmC levels decrease between two stages (i.e. %; WGBS) and the total level of 5hmC at both the stage immediately preceding and following this decrease. b) A model implicating 5mC oxidation in triggering DNA demethylation via an active mechanism predicts a positive correlation between the extent to which the combined 5mC/5hmC levels decrease between two stages (i.e. %; WGBS) and the relative 5hmC levels in the stage immediately preceding this decrease, as further oxidation of 5hmC to 5fC is the rate limiting step in the full oxidation of 5mC to 5caC³⁹. c) A model implicating oxidation of 5mC in safeguarding DNA hypomethylation following the major wave of DNA demethylation predicts that regions where the majority of DNA demethylation has been lost between two stages (i.e. those that are newly hypomethylated) will have high relative levels of 5hmC in the stage immediately following the major wave of DNA demethylation to remove residual methylation and/or aberrant de novo methylation. Thus, a limited correlation between the extent to which the combined 5mC/5hmC levels decrease between two stages (i.e. %, WGBS) and the relative 5hmC levels in the stage immediately following this decrease may also be seen.

FIG. 10—Tet1-3 expression and locus-specific DNA methylation in Tet1-KO PGCs during epigenetic reprogramming. a) Expression of Tet1 total transcript (left) or deleted exon 4 (right) in E12.5 Tet1-KO and wild type PGCs. Adjusted p-values (left) computed by DESeq2 and p-values (right) computed by Student's t-test. Asterisks refer to mean values. b) Representative immunostaining against the N-terminus of Tet1 protein in E12.5 wild type and Tet1-KO PGCs. Scale bar represents 10 μm. c) Expression of Tet2 and Tet3 in E12.5 Tet1-KO and wild type PGCs. Adjusted p-values computed by DESeq2. Asterisks refer to mean values. d-e) Mean combined 5hmC/5mC levels levels (RRBS) in female (d) or male (e) E12.5 and E14.5 Tet1-KO and wild type PGCs for ICRs and germline gene promoters called hypermethylated in E14.5 Tet1-KO PGCs. The mean DNA modification level and p-values were computed by RnBeads software (see Methods for details). f-g) Locus-specific bisulphite sequencing of the Dazl promoter (left), the Peg3 ICR (middle) and the IG-DMR ICR (right) in E12.5 (f) and E13.5 (g) female Tet1-KO and wild type PGCs. Specific details regarding sample sizes and how samples were collected are found in Statistics and Reproducibility section.

FIG. 11—Promoter DNA methylation clustering analysis during germline reprogramming. a) The combined promoter 5mC/5hmC levels (WGBS, right), promoter 5hmC levels (AbaSeq, centre), or gene expression levels (RNA-Seq, right) in consecutive stages of PGC development for all genes grouped by K-means clustering of the combined 5mC/5hmC dynamics at their promoter regions. b-c) Boxplots depicting the combined promoter 5mC/5hmC levels (WGBS, right), promoter 5hmC levels (AbaSeq, centre), or gene expression levels (RNA-Seq, right) in consecutive stages of PGC development for three clusters of genes with either low CpG promoters (LCPs; b) or intermediate CpG promoters (ICPs; c) grouped by K-means clustering of the combined 5mC/5hmC dynamics at their promoter regions. For all boxplots, the upper and lower hinges correspond to the first and third quartiles, the middle line corresponds to the median, and the maxima and minima respectively correspond to the highest or lowest value within 1.5× the inter-quartile range. Specific details regarding sample sizes and how samples were collected are found in Statistics and Reproducibility section.

FIG. 12—DNA modification and expression dynamics in wild type and Tet1-KO PGCs at retrotransposons normally activated concurrent with epigenetic reprogramming. a-b) Combined 5mC/5hmC dynamics in wild type PGCs (%; WGBS; far left), relative 5hmC dynamics (AbaSeq read counts normalised to E10.5; centre left) in wild type PGCs, the expression dynamics in either wild type or Tet1-KO PGCs (transcripts per million (TPM); RNA-Seq; centre right), and combined 5mC/5hmC dynamics in wild type and Tet1-KO PGCs (%; RRBS; far right) for representative repetitive elements significantly up-regulated (adj. p-value<0.05; Sleuth) in a sex-independent manner (a), in a male-specific manner (b, blue box), or in a female-specific manner (b, pink box) between E10.5 and E14.5 in wild type PGCs. Mean values are shown in all cases. Adjusted p-values for differential repeat expression analysis between E14.5 wild type and Tet1-KO PGCs are based on Sleuth software. Specific details regarding sample sizes and how samples were collected are found in Statistics and Reproducibility section.

FIG. 13—Characterisation of GRR gene regulation by Tet1 and 5mC in PGCs and mESCs. a) CpG density at GRR gene promoters and other relevant promoters; p-values are based on a two-sided Wilcoxon test. b) Mean 5hmC dynamics at GRR gene promoters and non-activated methylated and demethylating HCPs in PGCs; p-values are based on a two-sided paired Wilcoxon test. c) Log 2-fold change between Tet1-KO and wild type E14.5 male (blue) or female (pink) PGCs for GRR genes and other relevant gene sets. FWER-adjusted p-values are based on GSEA software (see Methods for details). d) Log 2-fold change between Dnmt1-CK0²⁴ and wild type mESCs (green), or between E14.5 female (pink) or male (blue) wild type PGCs and E10.5 wild type PGCs, for GRR genes and other relevant gene sets. FWER-adjusted p-values are based on GSEA software (see Methods for details). e) Correlation between the difference in combined 5mC/5hmC levels (x-axis; Tet1-KO (RRBS; %) -WT (RRBS; %)) at GRR promoters and the change in GRR gene expression (y-axis; log 2(Tet1-KO/WT)) in E12.5 (right) and E14.5 (left) Tet1-KO PGCs. Spearman correlation is shown. f) Representative western blot showing Tet1 and Lamin B protein expression in wild type, Dnmt-TKO, and Tet1-KO Dnmt-TKO mESCs. For all boxplots, the upper and lower hinges correspond to the first and third quartiles, the middle line corresponds to the median, and the maxima and minima respectively correspond to the highest or lowest value within 1.5× the inter-quartile range. For all figures, specific details regarding sample sizes and how samples were collected are found in Statistics and Reproducibility section. Specific details regarding sample sizes and how samples were collected are found in Statistics and Reproducibility section.

FIG. 14—Epigenetic characterisation of GRR gene promoters in mESCs. a) Genomic sequences centred on TSSs of GRR genes, non-GRR genes activated in both male and female PGCs between E10.5 and E14.5, and non-GRR methylated and demethylating HCP genes in wild type mESCs grown in serum-containing media. Each horizontal line represents one gene; the intensity of red indicates the relative enrichment for the feature shown at the top of each column. The TSS and sequences 5 kb upstream and downstream of the TSS are shown. b-f) Boxplots depicting the levels of: (b) combined 5mC/5hmC levels (WGBS)³; (c) 5hmC (AbaSeq)¹⁵; (d) Tet1 (ChIP-Seq)²¹; (e) Ring1b (ChIP-Seq)³⁸ and (f) H2Aub levels (ChIP-Seq)³⁷ at promoters of either GRR genes and other relevant gene sets in wild type mESCs grown in serum-containing media. For all boxplots, the upper and lower hinges correspond to the first and third quartiles, the middle line corresponds to the median, and the maxima and minima respectively correspond to the highest or lowest value within 1.5× the inter-quartile range. p-values are based on two-sided Wilcoxon test. g) Metagene plot depicting median H3K4me3 levels (ChIP-Seq)³⁰ around TSS of GRR genes (left) and non-GRR HCP genes that are also initially methlylated and subsequently demethylated during PGC reprogramming (right) in wild type and Tet1-KO mESCs grown in serum containing media. p-values are based on paired two-sided Wilcoxon test for region of TSS −1 kB/+500 bp. Specific details regarding sample sizes and how samples were collected are found in Statistics and Reproducibility section.

FIG. 15—Characterisation of GRR gene regulation by PRC1 and 5mC in PGCs and mESCs. a) Overlap between GRR genes and genes significantly up-regulated in E11.5 and/or E12.5 PRC1 conditional knockout PGCs compared with wild-type²⁶. p-values based on hypergeometric test. b) Representative western blot showing H2Aub and H2A levels in wild type or Dnmt-TKO mESCs+6h DMSO, and wild type or Dnmt-TKO mESCs+6h PRT4165 (PRC1 inhibitor). c) Classification of GRR genes depending on their dependency for 5mC and/or PRC1 reprogramming in mESCs (see Methods for details). Specific details regarding sample sizes and how samples were collected are found in Statistics and Reproducibility section.

FIG. 16—Model of endogenous PGC-to-gonocyte transition

The timely and efficient activation of germline reprogramming responsive (GRR) genes, involved in the PGC-to-gonocyte transition and successful gametogenesis, requires the interplay between the initiation of global DNA demethylation, Tet1 recruitment, and removal of PRC1-mediated repression. Both DNA demethylation-dependent (safeguarding against aberrant residual/de novo promoter DNA methylation) and -independent (such as the potential recruitment of OGT to gene promoters36, thus facilitating deposition of H3K4me3 via SET1/COMPASS38) functions of Tet1 are important for GRR gene activation.

FIG. 17—Change in gene expression in response to retinoic acid

Mouse embryonic stem cells (mESCs) were treated with retinoic acid (RA). The J1 cell line was used in comparison to J1 “TKO” cells lack DNA methylation machinery by virtue of being Dnmt1/Dnmt3a/Dnmt3b triple knock-out. The black bars respectively show the fold-change in Dazl-Hormad1- and Mae1-expression in TKO cells compared with J1 controls (neither treated with RA). The grey bars respectively show the fold-change in Dazl-Hormad1- and Mae1-expression in J1 cells treated with RA compared with J1 cells not treated with RA. The white bars respectively show the fold-change in Dazl-Hormad1- and Mae1-expression in TKO cells treated with RA compared with J1 cells not treated with RA.

### DETAILED DESCRIPTION OF THE PREFERRED EMBODIMENTS AND FURTHER OPTIONAL FEATURES OF THE INVENTION

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

Epigenetic Reprogramming Enables the Primordial Germ Cell-to-Gonocyte Transition

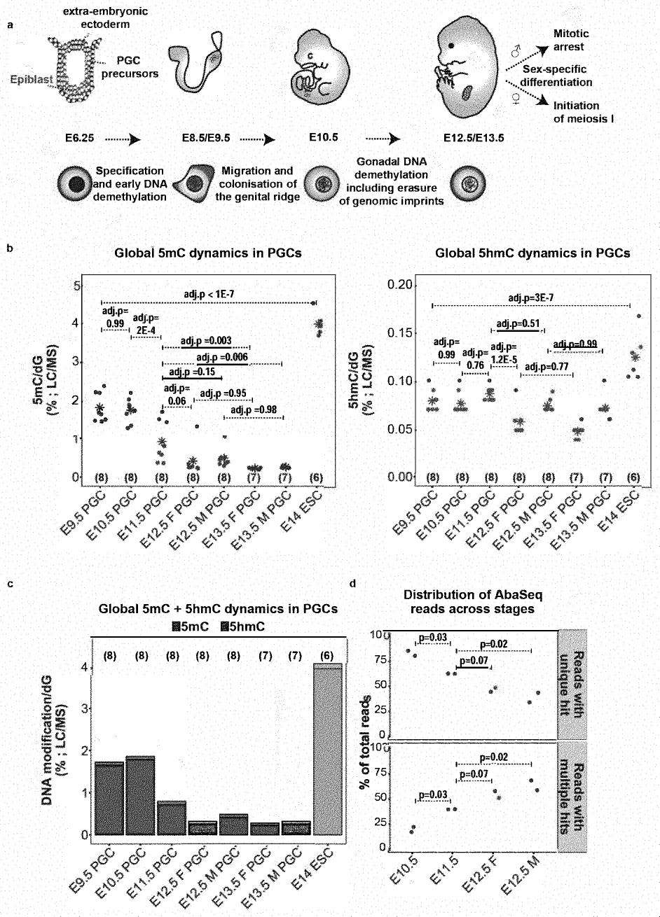

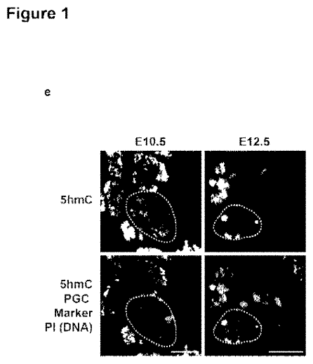

Gametes are highly specialised cells that can give rise to the next generation through their ability to generate a totipotent zygote. In mouse, germ cells are first specified in the developing embryo as primordial germ cells (PGCs) starting around embryonic day (E) 6.25¹ (FIG. 1a). Following subsequent migration into the developing gonad, PGCs undergo a wave of extensive epigenetic reprogramming at E10.5/E11.5²⁻¹¹, including genome-wide loss of 5-methylcytosine (5mC)^{2-5,7-11} (FIG. 1a). The underlying molecular mechanisms of this process have remained enigmatic leading to our inability to recapitulate this step of germline development in vitro¹²⁻¹⁴. Using an integrative approach, the inventors show that this complex reprogramming process involves the coordinated interplay between promoter sequence characteristics, DNA (de)methylation, Polycomb (PRC1) complex and both DNA demethylation-dependent and -independent functions of Tet1 to enable the activation of a critical set of germline reprogramming responsive (GRR) genes involved in gamete generation and meiosis. Our results also unexpectedly reveal a role for Tet1 in safeguarding but not driving DNA demethylation in gonadal PGCs. Collectively, our work uncovers a fundamental biological role for gonadal germline reprogramming and identifies the epigenetic principles of the PGC-to-gonocyte transition that will be instructive towards recapitulating complete gametogenesis in vitro.

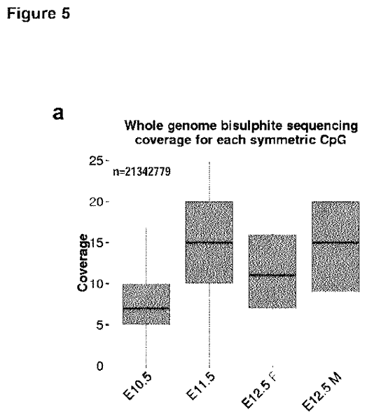

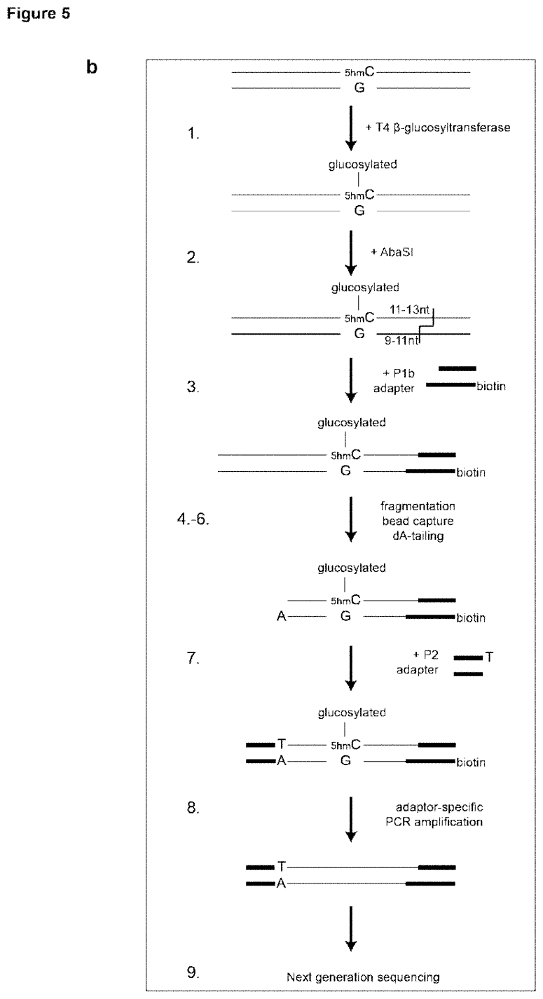

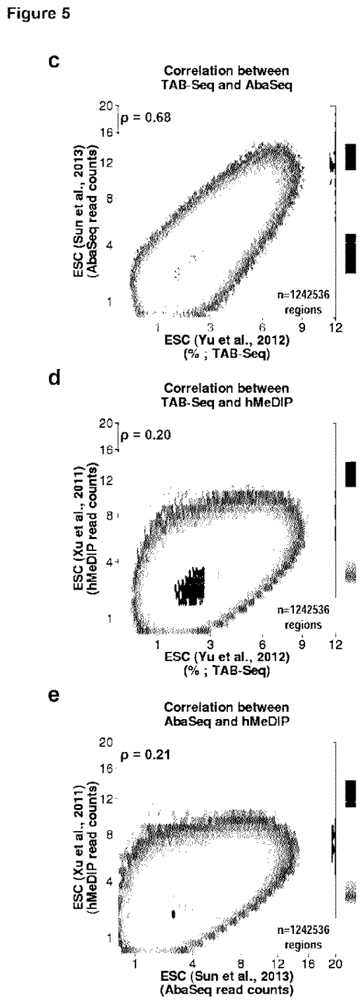

In order to address the potential role and underlying molecular mechanisms of gonadal germline reprogramming, the inventors first set out to investigate the dynamics of and relationship between 5mC and 5-hydroxymethylcytosine (5hmC), which has previously been implicated in DNA demethylation in PGCs^{3,6,9-11}. The inventors did this quantitatively and at single base resolution using liquid chromatography/mass spectrometry (LC/MS) coupled with whole genome bisulphite sequencing (WGBS, FIG. 5a) and AbaSeq¹⁵ (FIG. 5b-e). WGBS provides information regarding combined levels of 5mC and 5hmC¹⁶, while AbaSeq¹⁵ enables robust site-specific quantification and accurate comparison of 5hmC levels genome-wide within a given sample and between samples when combined with LC/MS (see Methods, FIG. 5b-e).

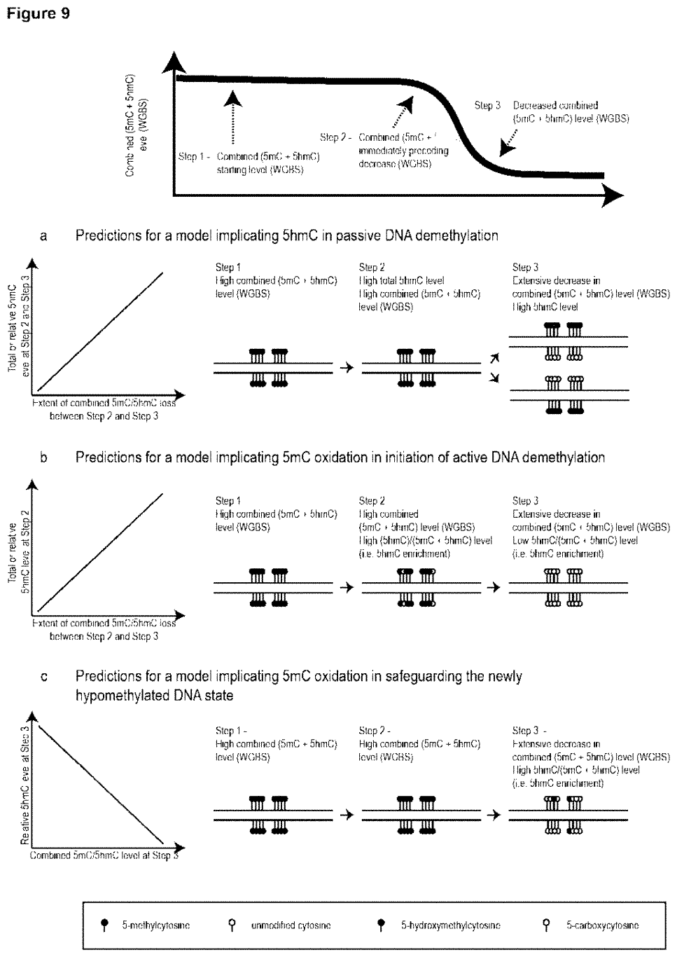

By LC/MS the inventors observed that global levels of genomic 5mC remain stable between migratory (E9.5) and early gonadal (E10.5) PGCs, followed by a significant reduction between E10.5 and E11.5 and much more limited DNA demethylation between E11.5 and E13.5 (FIG. 1b). With respect to 5hmC, LC/MS analysis surprisingly revealed that global levels in PGCs are lower than those in mouse embryonic stem cells (mESCs) grown in serum-containing culture conditions (FIG. 1b). Furthermore, the global 5hmC levels in PGCs are relatively constant between E9.5 and E13.5, with a slight decrease in females starting at E12.5 (FIG. 1b). Importantly, 5hmC levels are consistently an order of magnitude lower than either total 5mC levels at E10.5 or the amount of 5mC lost between E10.5 and E11.5 (FIG. 1b-c), documenting that DNA demethylation is globally not accompanied by a reciprocal increase in 5hmC levels, as has previously been suggested^{3,17} (FIG. 9a).

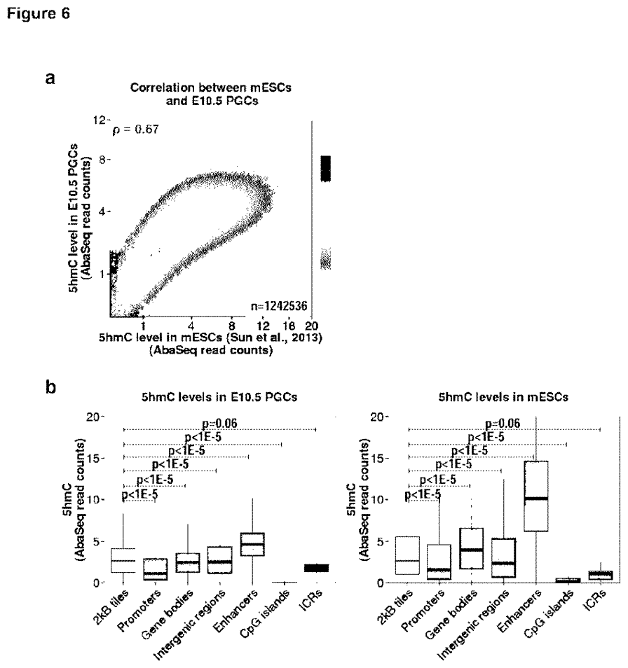

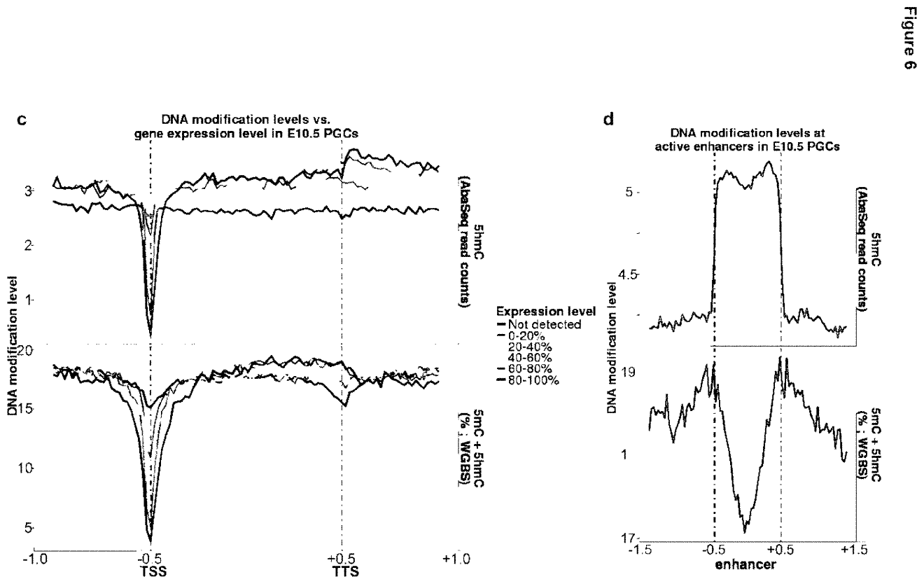

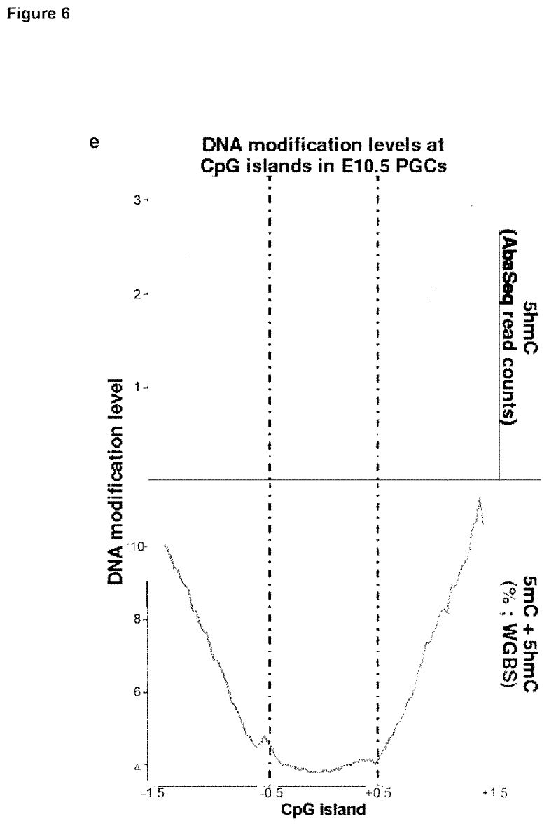

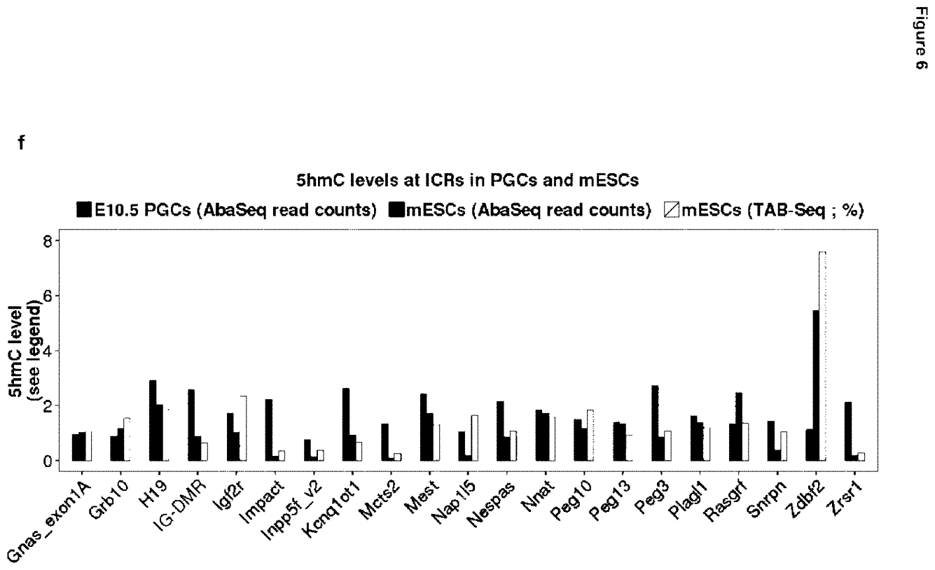

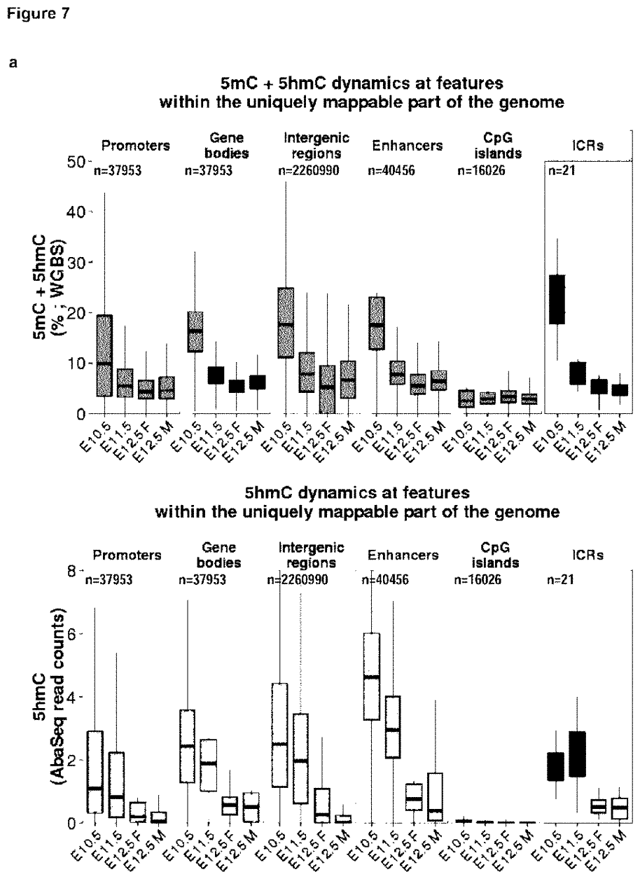

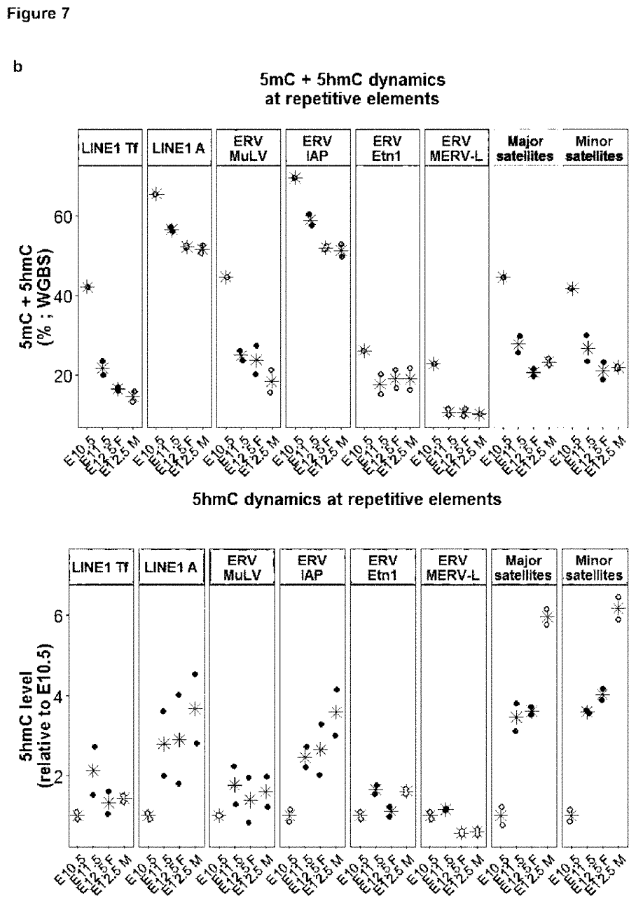

Consistent with our LC/MS measurements, WGBS analysis revealed near complete loss of combined 5mC/5hmC between E10.5 and E11.5 at features within uniquely mapped regions of the genome, with limited further DNA demethylation observed between E11.5 and E12.5 (FIG. 7a). Loss of DNA methylation was also observed at consensus repeat sequences, although some repetitive elements such as LINE-1A and ERV-IAP retrotransposons retained comparatively high levels of combined 5mC/5hmC in E12.5 PGCs, as previously suggested⁸ (FIG. 7b). Detailed analysis of 5hmC localisation by AbaSeq in E10.5 PGCs revealed that, although global levels are lower (FIG. 1b), 5hmC localisation in PGCs is remarkably similar to that of serum-grown mESCs, even at imprint control regions (ICRs; FIG. 6a,b,f). Overall, 5hmC was enriched at putative active enhancers, present in intergenic regions and gene bodies, depleted at promoters, and absent on the vast majority of CpG islands (FIG. 6b-f). With respect to transcription, both 5mC and 5hmC at promoter regions show an inverse relationship with gene expression levels (FIG. 6c). Within gene bodies, 5mC and 5hmC are clearly enriched at expressed genes compared to genes without detectable expression, although a non-linear relationship with gene expression is observed for 5hmC while combined 5mC/5hmC levels show a clear positive correlation (FIG. 6c).

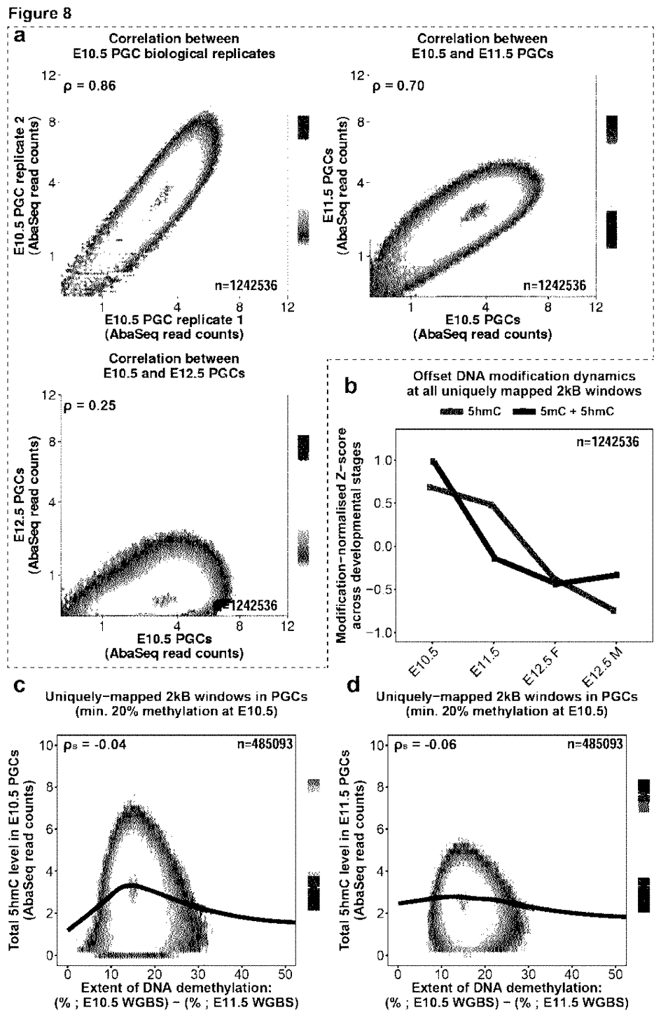

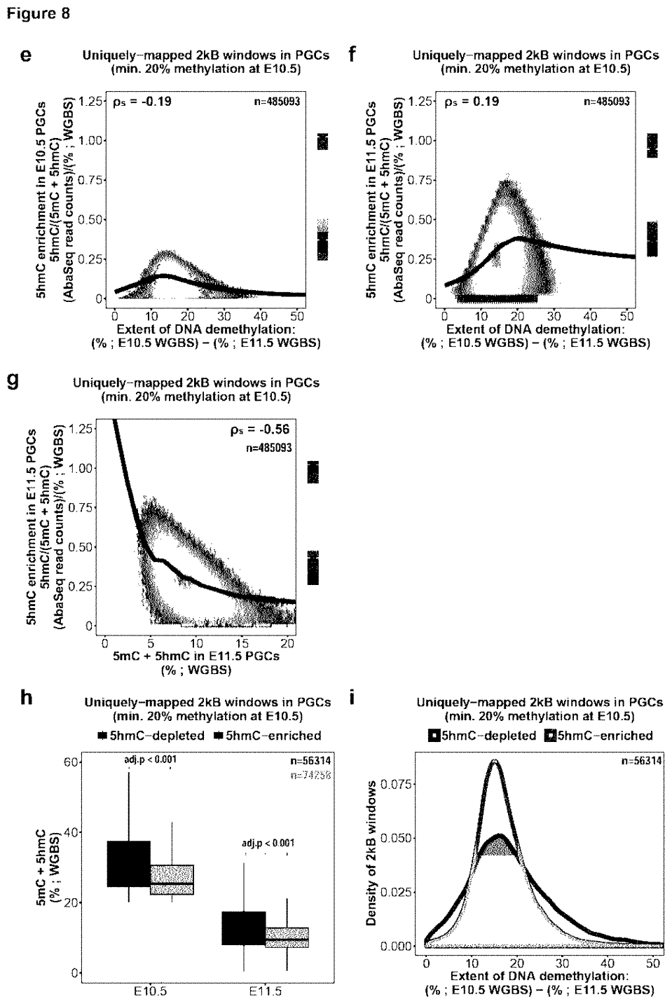

Detailed analysis of 5hmC patterns across examined developmental stages uncovered that the majority of 5hmC is lost from uniquely mapped regions of the genome and re-localised to repetitive elements (FIG. 1d, FIG. 7a-b). This relocalisation was also clearly evident by immunofluorescence staining (FIG. 1e). Our data thus shows that both 5mC and 5hmC are lost in PGCs throughout the uniquely mapped regions of the genome, although with different kinetics with 5hmC showing a more gradual decrease (FIG. 8b). However, this was not consistent with passive dilution of 5hmC through cell divisions³ as demonstrated by poor Pearson and Spearman correlations between stages (FIG. 8a, 9a). To the contrary, the inventors conclude that 5hmC is a dynamic mark in PGCs.

We next explored the relationship between 5hmC deposition and DNA demethylation in gonadal PGCs between E10.5 and E12.5 for all initially methylated 2 kb windows (i.e. min. 20% methylation at E10.5). DNA demethylation involving a 5hmC intermediate predicts a direct correlation between 5hmC appearance and 5mC loss (FIG. 9a-b). To the inventors' surprise, no correlation between either the total or relative 5hmC levels at E10.5 or E11.5 was observed, and the extent that the combined 5mC/5hmC levels decrease between these stages (FIG. 8c-f). However, for all initially methylated 2 kb windows, a negative correlation between the relative 5hmC level and the combined 5mC/5hmC levels at E11.5 is observed (FIG. 8g). Thus, 5hmC represents a much higher proportion of combined 5mC/5hmC at regions that are newly hypomethylated at E11.5, regardless of their original DNA methylation levels. Although 5hmC-depleted regions contain slightly more 5mC at E11.5 than regions enriched for 5hmC, sequences depleted of 5hmC in both E10.5 and E11.5 PGCs still undergo considerable DNA demethylation between these two stages (FIG. 8h-i), indicating that the presence of detectable 5hmC is not a prerequisite for 5mC loss in gonadal PGCs. Our observations thus implicate involvement of 5hmC in the regulation of the post-DNA demethylation locus-specific 5mC levels in germ cells rather than in the initial wave of global DNA demethylation (FIG. 9c).

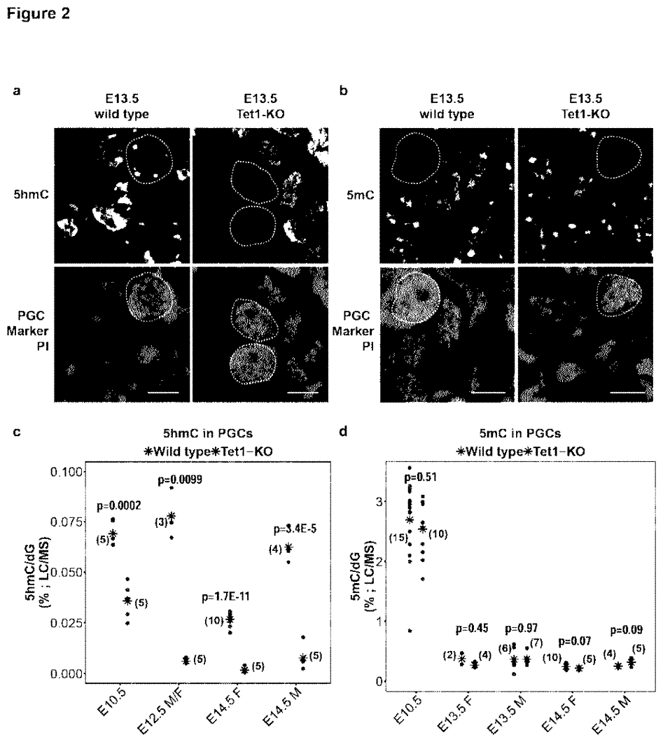

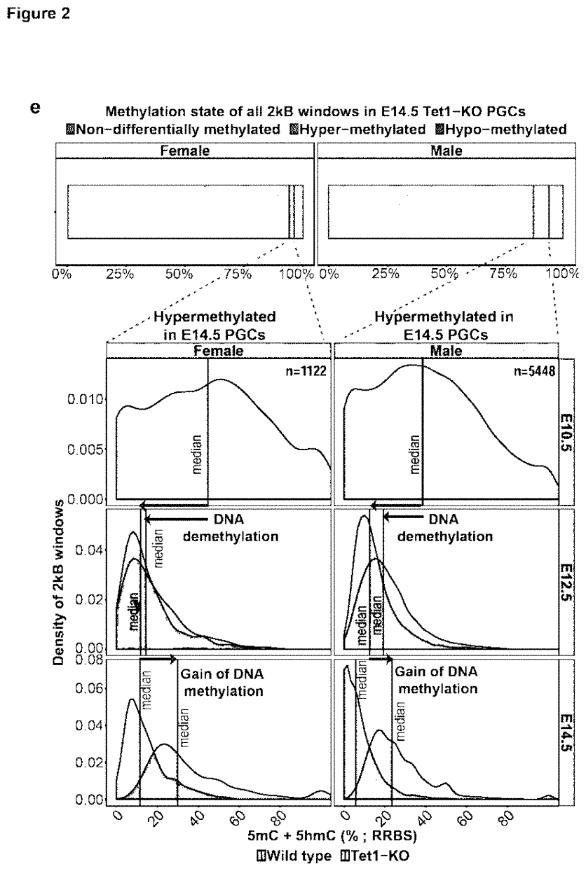

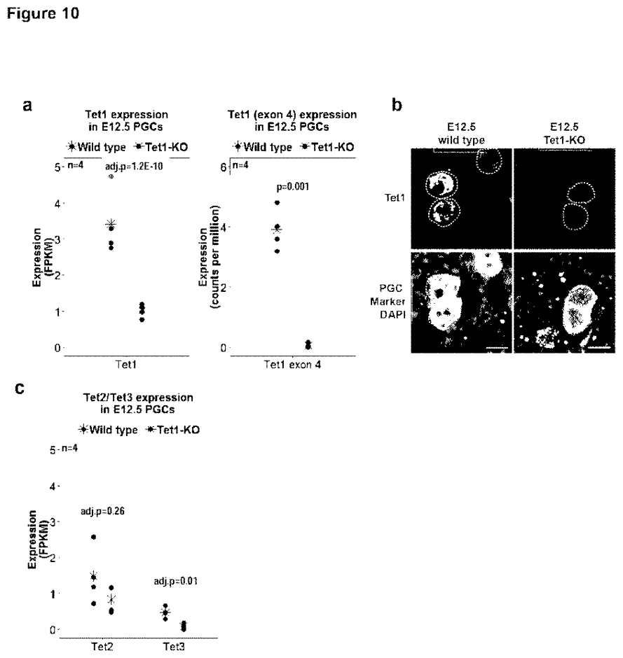

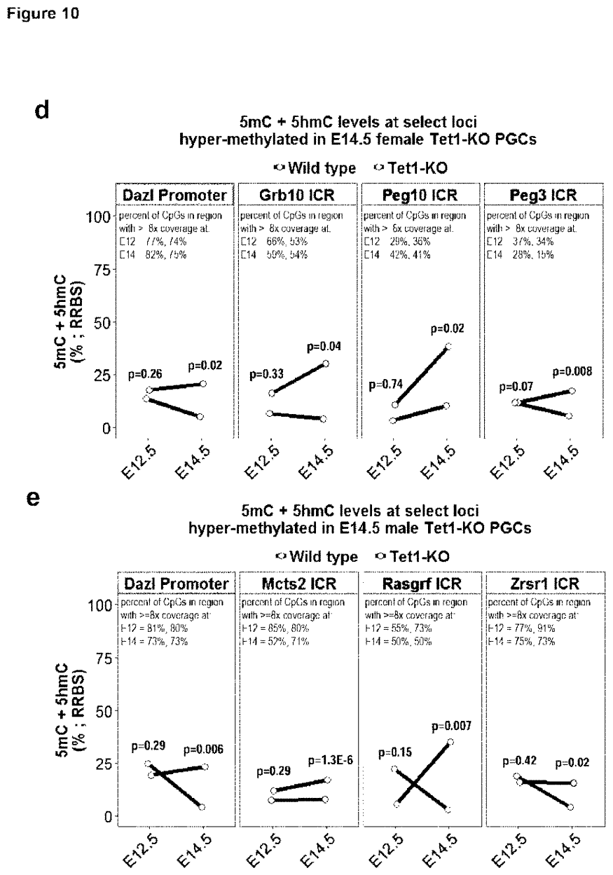

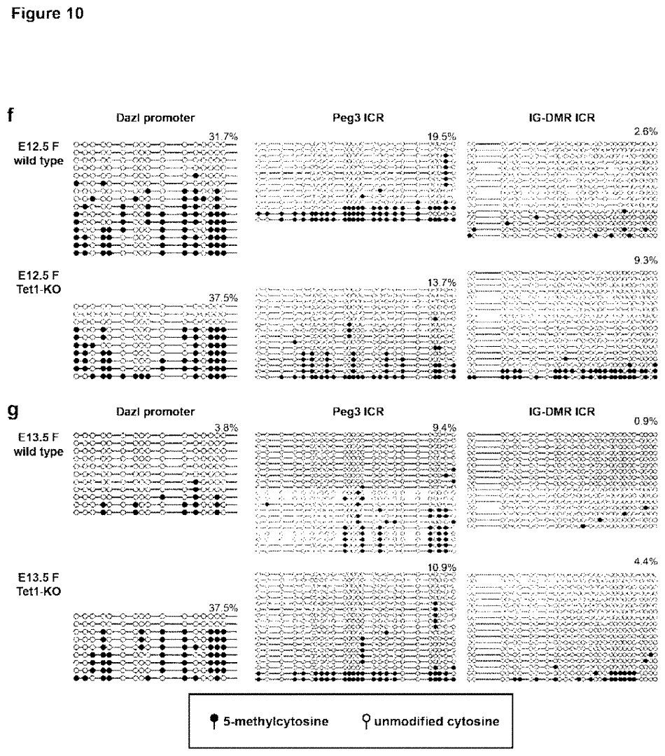

To expand on this observation, the inventors used a previously published Tet1-KO mouse model¹⁸ (FIG. 10a-c). Initial LC/MS analysis revealed that loss of Tet1 leads to approximately 50% reduction in global 5hmC levels in E10.5 Tet1-KO germ cells (FIG. 2c). In agreement with the high level of Tet1 expression at E12.5^{3,9,11} (FIG. 10a-c), LC/MS analysis confirmed that Tet1 represents the primary 5mC oxygenase in demethylated PGCs, with approximately 85% decrease in global 5hmC levels observed in E14.5 Tet1-KO germ cells (FIG. 2a,c). Importantly, the genome of both Tet1-KO and wild type PGCs reached near-complete depletion of 5mC by E13.5 (FIG. 2b,d), highlighting that Tet1-mediated 5mC oxidation is not directly responsible for the bulk of DNA demethylation in gonadal PGCs.

In support of our LC/MS measurements, only a limited number of differentially methylated regions were detected in E14.5 Tet1-KO PGCs by reduced representation bisulphite sequencing (RRBS) (FIG. 2e). Intriguingly, these regions initially undergo extensive DNA demethylation in both Tet1-KO and wild type PGCs, followed by a subsequent increase in 5mC levels specifically in Tet1-KO PGCs between E12.5 and E14.5 (FIG. 2e). In contrast, 5mC levels remain stable and/or undergo slight further reduction between these stages in wild type germ cells (FIG. 2e). The same DNA demethylation/remethylation kinetics were also observed at the few examples of previously reported^{9,10} germline gene promoters and ICRs that were found hypermethylated in E14.5 Tet1-KO PGCs by RRBS (FIG. 10d-e). Although significant enrichment of 5mC is indeed observed at the Dazl promoter by targeted bisulphite sequencing in demethylated PGCs, the extent of hypermethylation observed at the Peg3 and IG-DMR ICRs is in fact very limited (FIG. 10f-g). Furthermore, for all three regions, very few clones retained full methylation while a number of clones had heterogeneous methylation patterns consistent with a stochastic failure to remove aberrant residual/de novo DNA methylation in Tet1-KO PGCs (FIG. 10f-g).

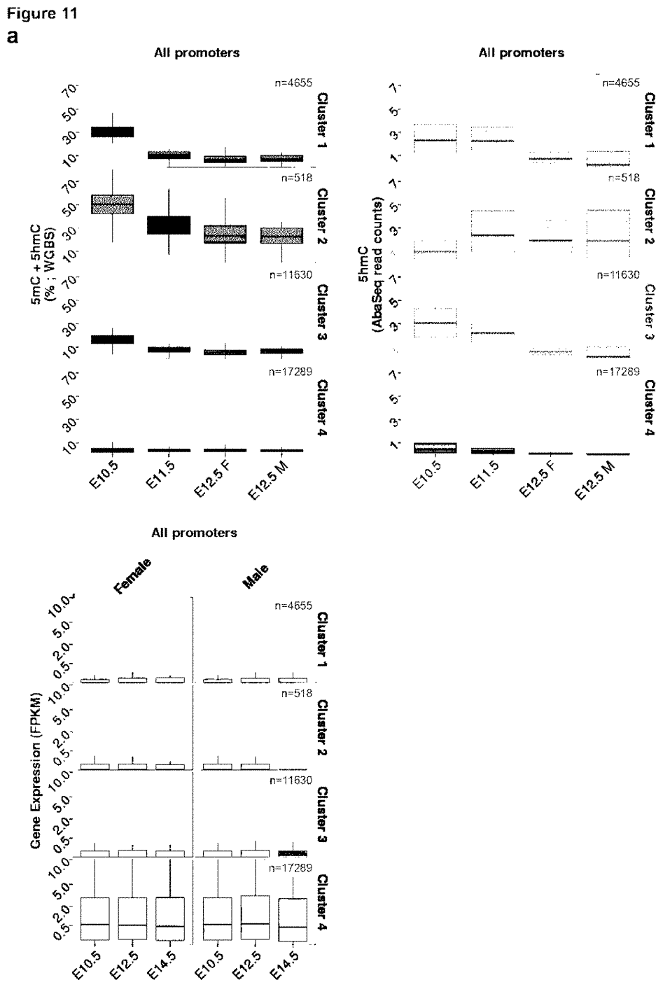

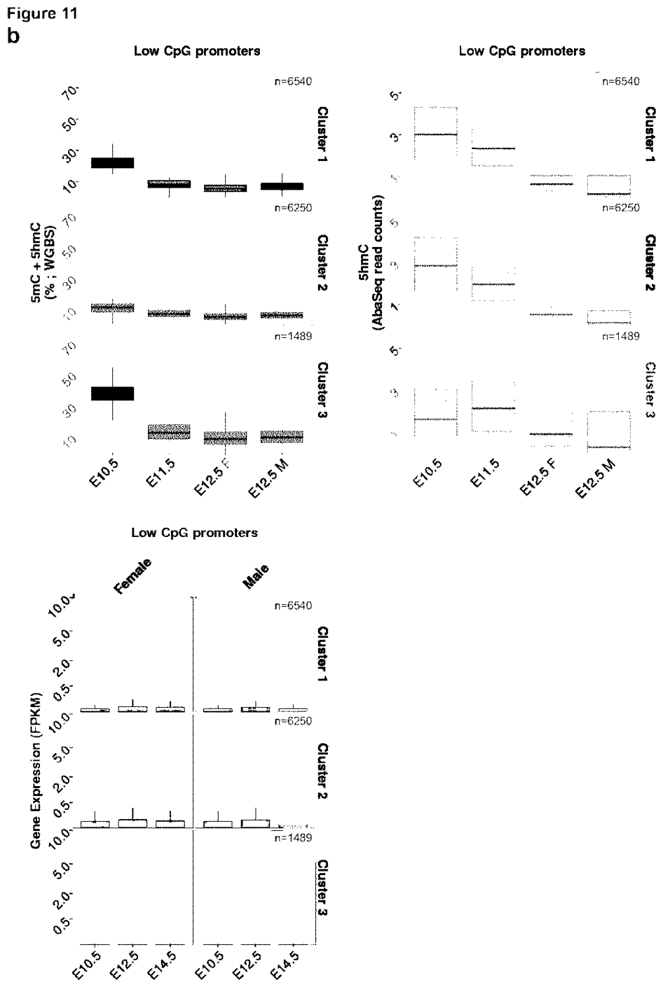

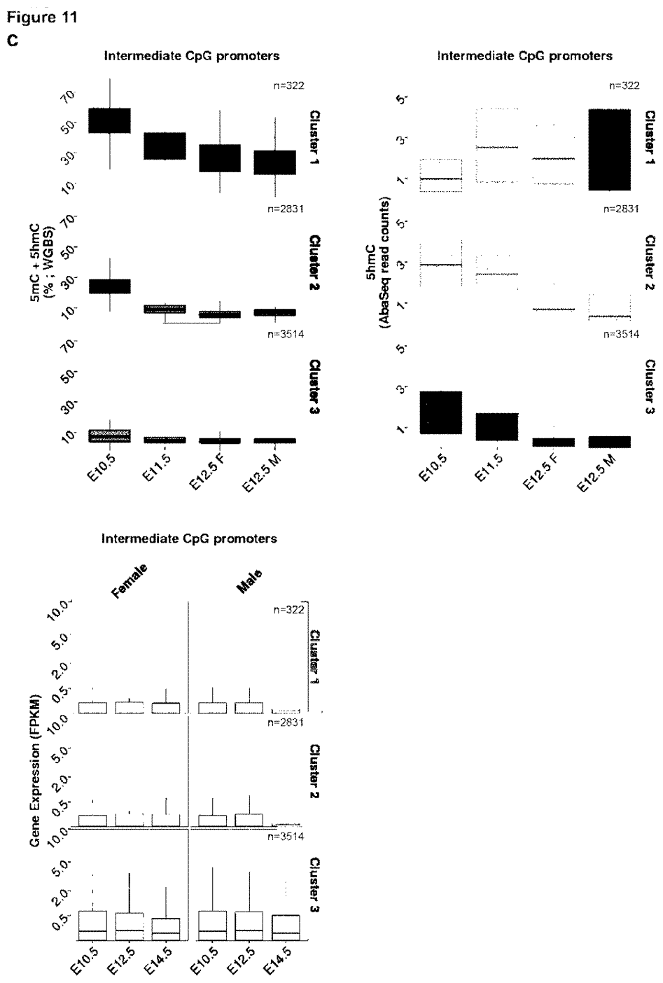

We next analysed the observed 5mC and 5hmC dynamics in combination with RNA-Seq datasets derived from E10.5-E14.5 PGCs (FIG. 11). Initial clustering analysis of all genes based on their promoter DNA methylation dynamics revealed that, while most promoters become completely demethylated, there is a small subset of transcriptionally silenced promoters that retain high levels of 5mC/5hmC during global DNA demethylation (cluster 2, FIG. 11a). These promoters significantly overlap with LINE1 and LTR (p-values=9.5×10⁻²⁴ and 7.2×10⁻⁸³ respectively, hypergeometric test) containing endogenous retroviruses that are likely to determine this epigenetic status (FIG. 7b). Overall, although high levels of promoter 5mC and 5hmC are associated with transcriptional repression in E10.5 pre-reprogramming PGCs, loss of these marks does not generally result in transcriptional activation (FIG. 11a).

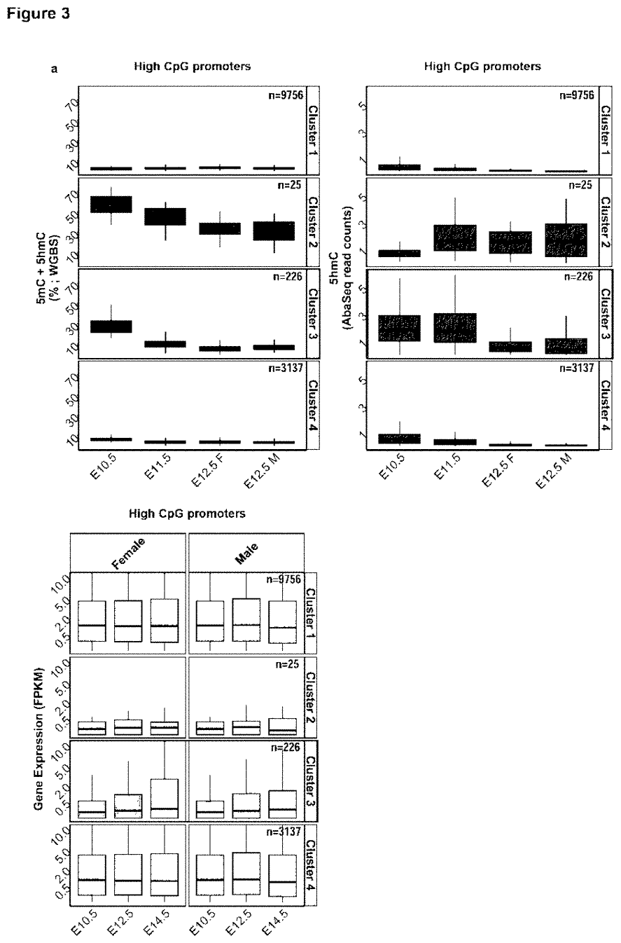

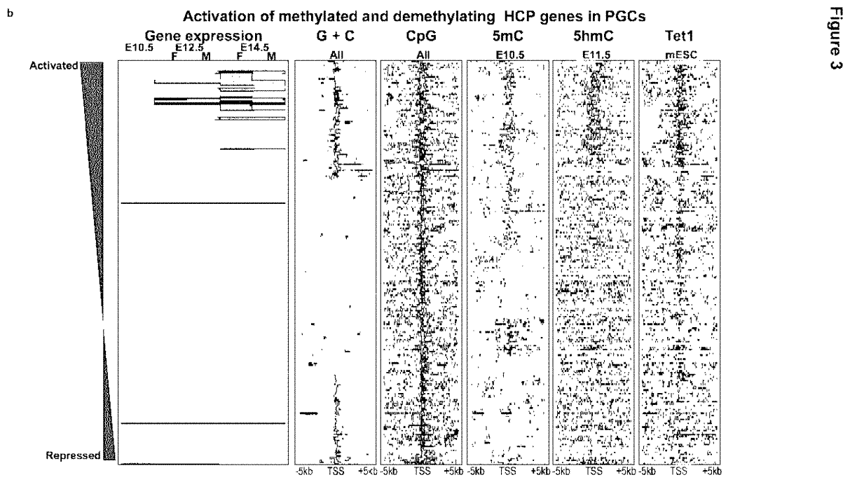

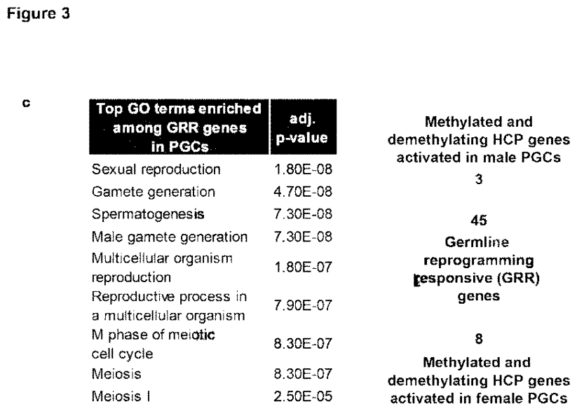

As the influence of 5mC on transcriptional activity of a gene has been shown in mammals to be highly dependent on promoter CpG content¹⁹, the inventors performed clustering analysis specifically at genes with either high-CpG (HCPs), intermediate-CpG (ICPs) or low-CpG (LCPs) promoters¹⁹ (FIG. 3a and FIG. 11b-c). Interestingly, this yielded a group of HCP genes that became DNA demethylated during the course of germline epigenetic reprogramming, and showed progressive transcriptional activation (cluster 3; FIG. 3a). Differential expression analysis confirmed that these genes show a significant enrichment among all genes up-regulated concurrent with epigenetic reprogramming in PGCs (p-value<0.001, hypergeometric test), with 45 genes commonly activated in both sexes (FIG. 3a-c). Considering their promoter methylation dynamics and timing of their activation, the inventors term these 45 genes ‘germline reprogramming responsive’ (GRR) genes (FIG. 3c). Interestingly, GRR genes shows significant enrichment for factors involved in gamete generation and meiosis, including Dazl, Sycp1-3, Mae1, Hormad1, and Rad51c (FIG. 3c).

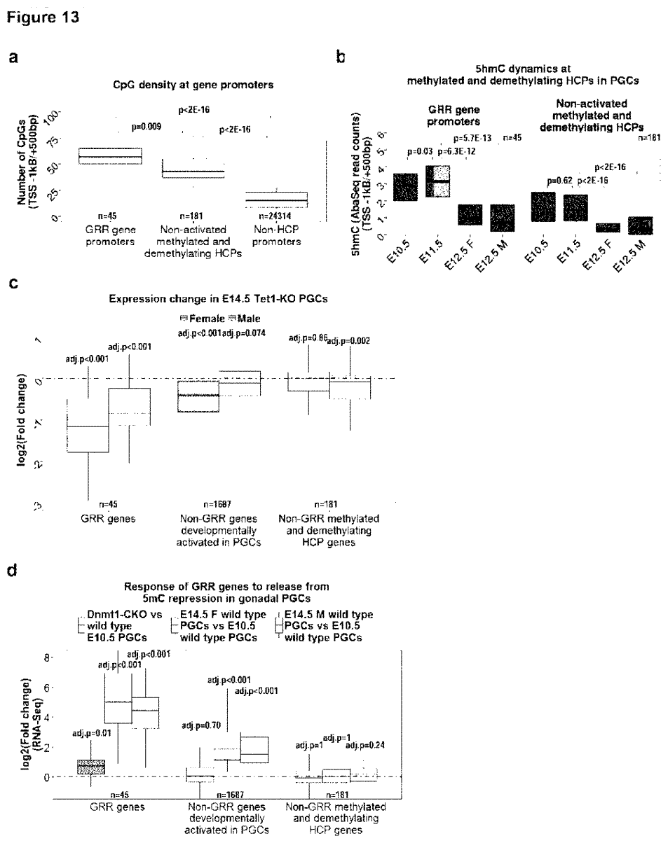

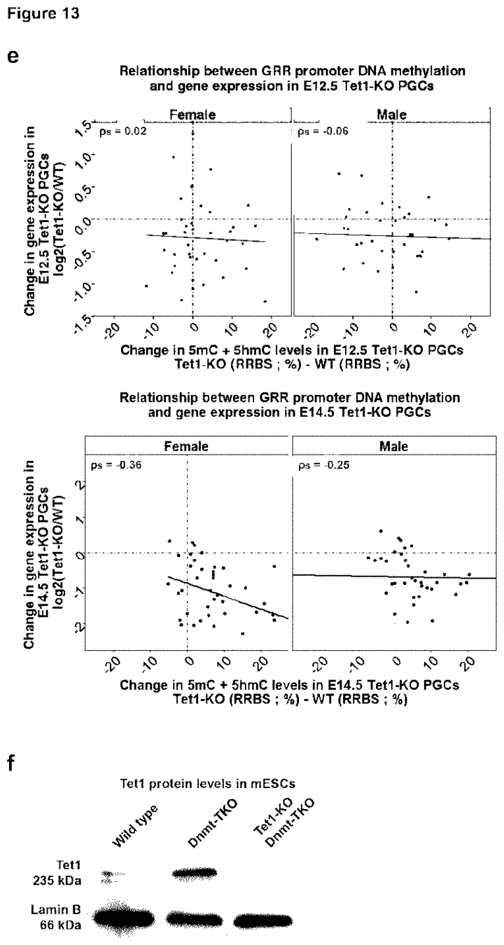

Considering that GRR genes (n=45) constituted less than 25% of the entire subset of HCP genes that undergo DNA demethylation (n=226; FIG. 3a-c), DNA demethylation is likely an important factor for transcriptional activation of methylated HCPs, with other factors additionally necessary. Indeed, GRR gene promoters showed both exceptionally high CpG density and 5hmC levels compared to other methylated and demethylating HCPs (FIG. 13a-b). It was also noted that, unusually for promoters, 5hmC levels transiently increased at GRR gene promoters in PGCs immediately following the major wave of DNA demethylation (FIG. 7a, 13b). In addition, and in agreement with their high CpG density and 5hmC levels^{20,21}, GRR gene promoters have been shown to be bound by Tet1 in both mESCs²¹ and PGCs⁹ (FIG. 3b).

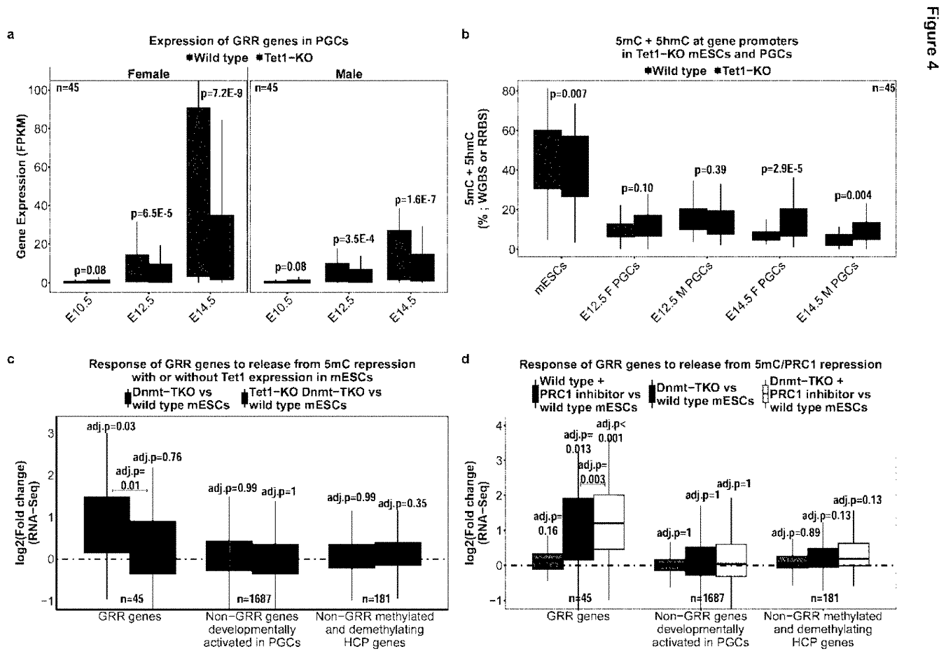

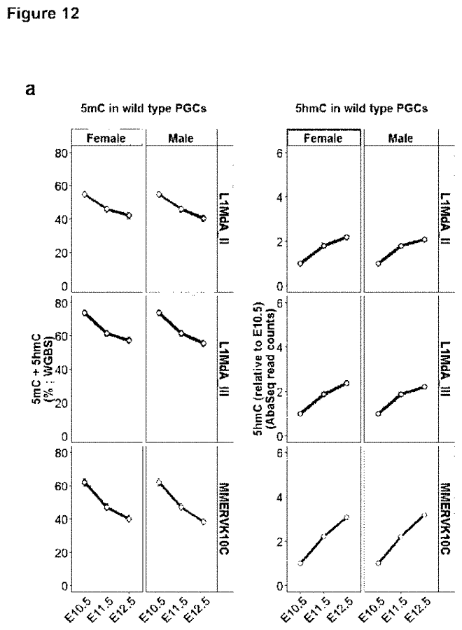

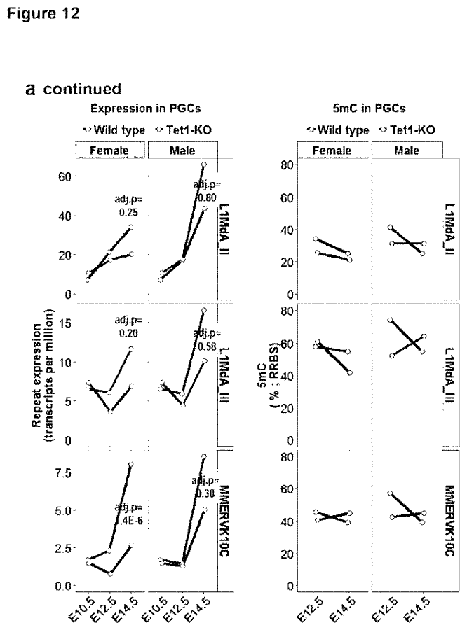

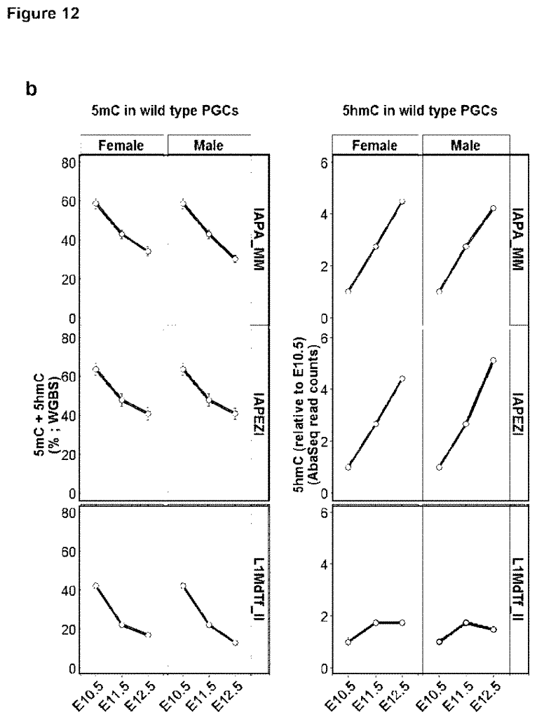

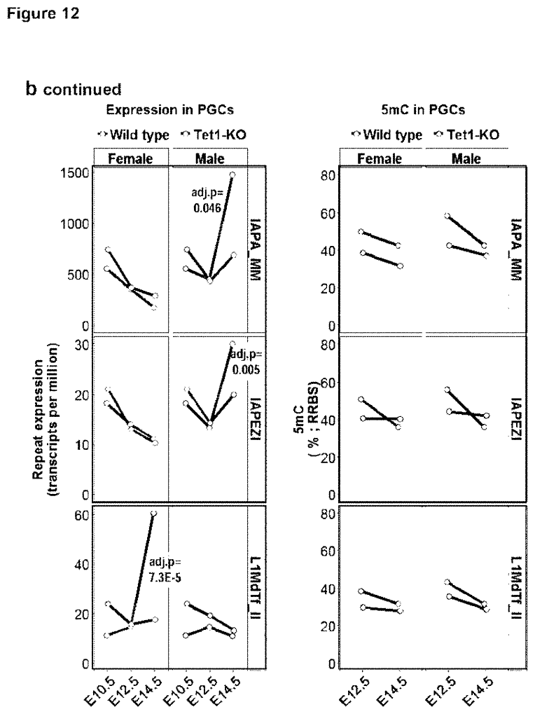

The observed binding of Tet1 is functionally relevant, as the extent of GRR gene upregulation is considerably lower in Tet1-KO PGCs (FIG. 4a, FIG. 13c). Although GRR gene promoters undergo normal DNA demethylation in the absence of Tet1 by E12.5, they show slight hypermethylation later in E14.5 Tet1-KO PGCs (FIG. 4b). However, this limited DNA hypermethylation shows only weak correlation with the decreased expression (FIG. 13e). Furthermore, lower expression of GRR genes in Tet1-KO germ cells is already apparent at E12.5 in the absence of any methylation differences (FIG. 4a-b, FIG. 13e), suggesting that Tet1 is potentially acting as a transcriptional regulator outside its role in 5mC removal^{21,22}. In addition to GRR genes, transposable elements (TE) show accumulation of 5hmC during gonadal epigenetic reprogramming (FIG. 7b, 12). Alongside reduction in DNA methylation, some TEs show transcriptional activation concurrent with epigenetic reprogramming, especially from evolutionary young retrotransposons (FIG. 12). Interestingly, the lack of Tet1 appears to also reduce the extent of transcriptional activation of normally activated TEs (FIG. 12).

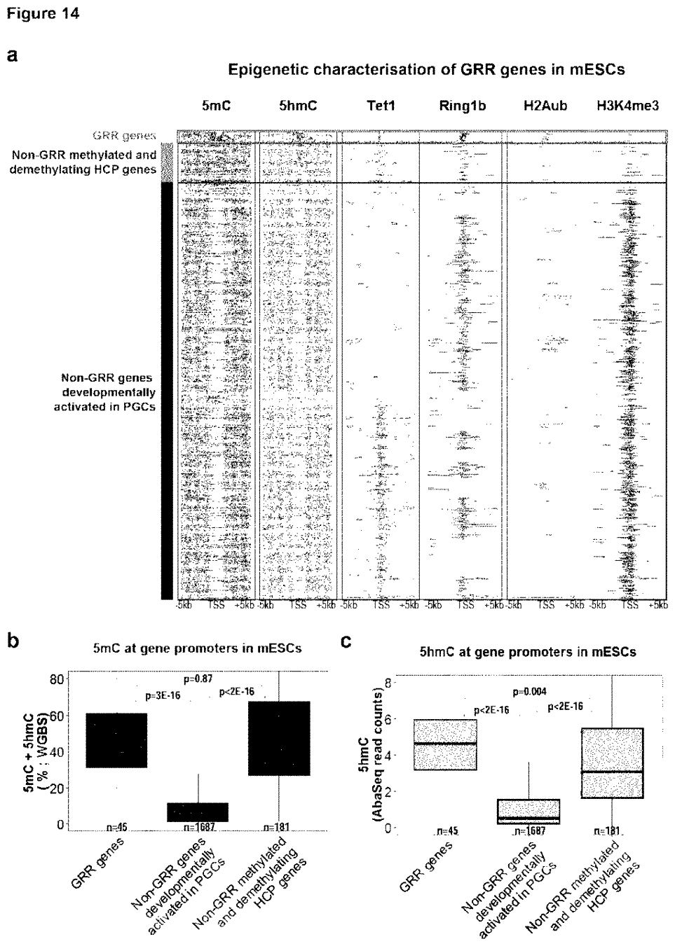

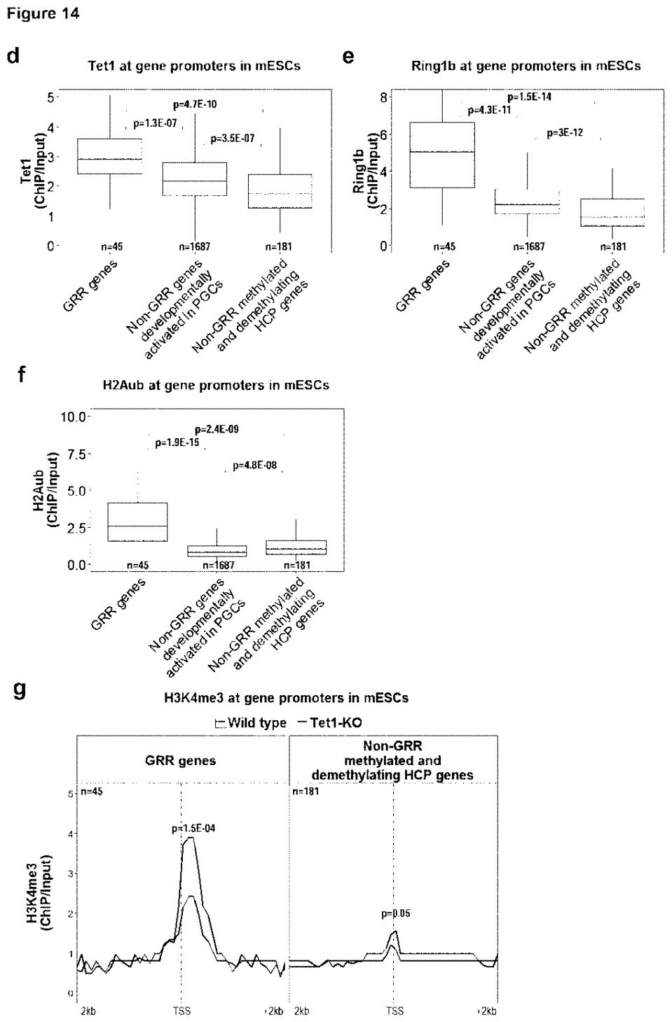

To further mechanistically probe the causal relationship between epigenetic reprogramming and GRR gene activation, the inventors turned to an in vitro model. Serum-grown mESCs represented an ideal system, as these cells are not germ line-restricted yet have highly similar epigenetic modifications at GRR gene promoters to what is observed in vivo in pre-reprogramming gonadal PGCs (FIG. 14a-d). Consistent with what the inventors observed in vivo, promoter DNA demethylation represents a dominant epigenetic reprogramming event for GRR gene activation also in vitro. Dnmt-TKO²³ mESCs display increased expression of GRR genes (FIG. 4c). However, even in the complete absence of DNA methylation, this is crucially dependent on the presence of Tet1 as Tet1-KO Dnmt-TKO mESCs fail to activate GRR genes as a group (FIG. 4c, FIG. 13f).

Although these in vitro observations clearly supported our in vivo data with respect to the roles of 5mC and Tet1, the extent to which GRR genes were up-regulated in Dnmt-TKO mESCs (FIG. 4c) or in E10.5 PGCs that have undergone precocious DNA demethylation by conditional deletion of Dnmt1 (Dnmt1-CKO)²⁴ (FIG. 13d) was relatively mild. The inventors thus hypothesised that other factors, including potentially other epigenetic barriers, may regulate GRR gene expression. In this context, gonadal epigenetic reprogramming has been previously linked with erasure of epigenetic information at various distinct levels^{4,25}, with removal of Polycomb Repressive Complex 1 (PRC1) previously shown to coordinate the timing of meiosis initiation in DNA demethylated E11.5/E12.5 PGCs²⁶. Remarkably, genes aberrantly up-regulated following PRC1 deletion in PGCs show a significant enrichment for GRR genes (FIG. 15a) and promoters of GRR genes in serum-grown mESCs are enriched for Ring1b binding and H2AK119ub (FIG. 14a,e,f). In view of this, the inventors simultaneously abolished both DNA methylation and PRC1 activity using highly specific chemical inhibition of PRC1 in the context of Dnmt-TKO mESCs to test the role of both DNA methylation and PRC1 in GRR gene regulation, thus mimicking gonadal epigenetic reprogramming. Culture of mESCs with PRT4165²⁷ resulted in significant inhibition of PRC1-mediated H2A ubiquitination after only 6h of culture (FIG. 15b). Dual inhibition of 5mC/PRC1 repression strikingly resulted in the activation of 33 out of 45 GRR genes with 25 and 10 genes activated upon the sole inhibition of either 5mC or PRC1 repression, respectively (FIG. 4d, FIG. 15c). Combined, these observations show that gonadal epigenetic reprogramming entails a composite erasure of epigenetic systems^{4,25} to potentiate the expression of GRR genes.

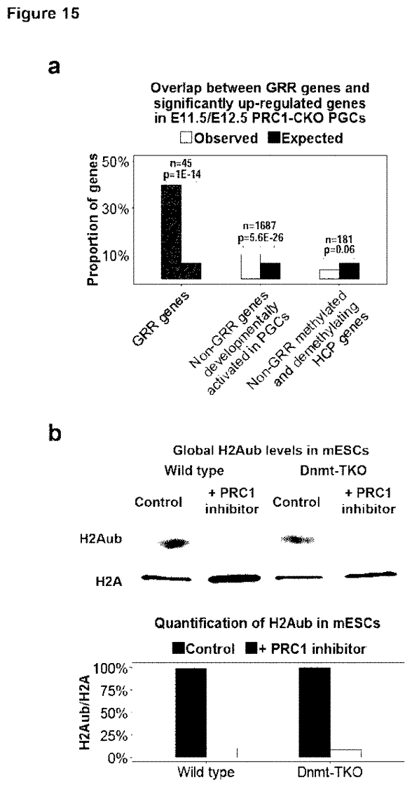

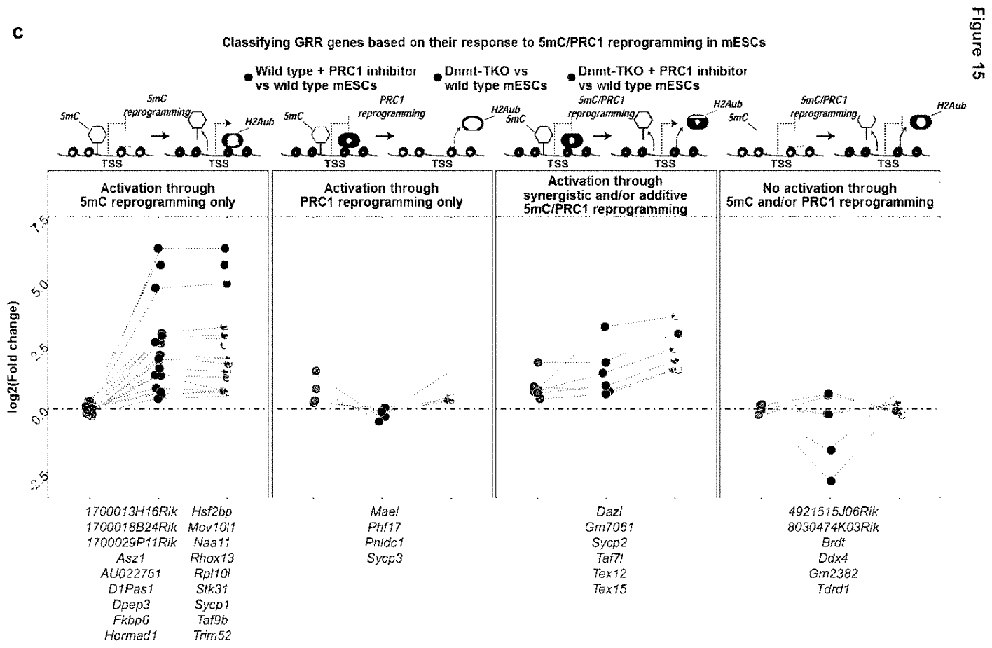

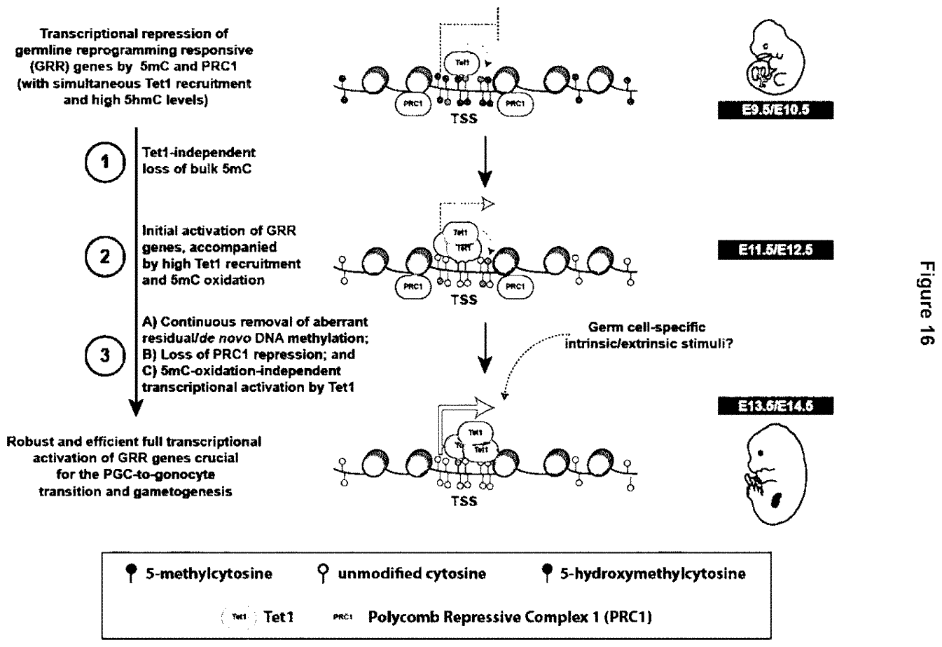

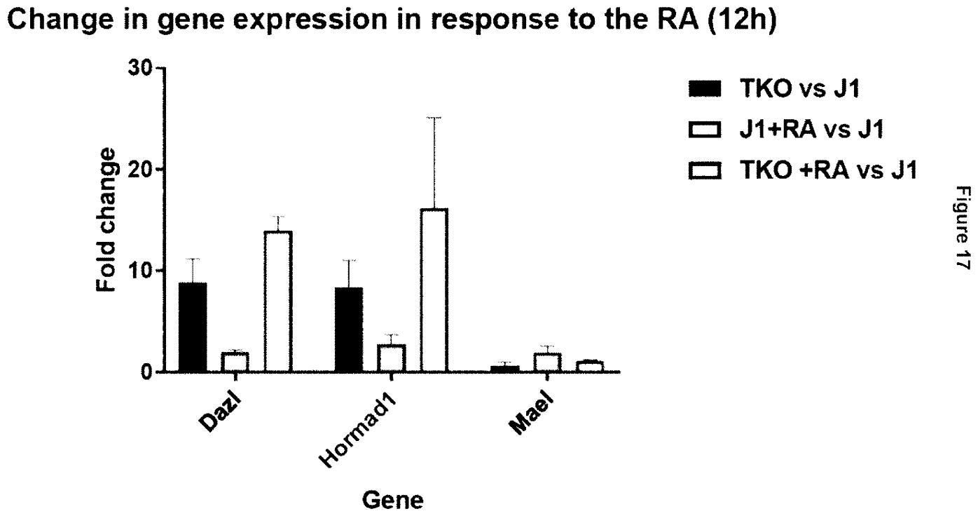

Our study has identified a set of germline reprogramming responsive (GRR) genes crucial for the correct progression of gametogenesis. These genes have unique promoter sequence characteristics, with high levels of both 5mC and 5hmC, and are targets of Tet1 and PRC1. This disclosure shows that combined loss of DNA methylation and PRC1 repression is uniquely required for GRR gene activation, with this epigenetically poised state further requiring Tet1 to potentiate both full and efficient activation. Tet1 appears to be particularly important in female PGCs⁹, which initiate meiotic prophase soon after completion of epigenetic reprogramming, thus posing a requirement on the timely high expression of these genes. Importantly, although the inventors observed slight hypermethylation at GRR gene promoters in E14.5 Tet1-KO PGCs, our study clearly documents that Tet1 stimulates transcription of GRR genes also via a DNA demethylation-independent mechanism^{21,22}. In this context, previous studies have shown that Tet1 recruits OGT to gene promoters²², thus facilitating deposition of H3K4me3 via SET1/COMPASS²⁸ leading to transcriptional activation. In further support, GRR gene promoters in mESCs are marked by low but detectable H3K4me3, the levels of which are significantly decreased in the absence of Tet1 without changes in DNA methylation (FIG. 4b, FIG. 14g). Tet1 may additionally potentiate transcription through regulation of 5mC/5hmC levels at non-promoter cis-elements, such as enhancers. Last, but not least, our study shows that Tet1 is not directly involved in initiation of global DNA demethylation during epigenetic reprogramming in gonadal PGCs, but rather the inventors define a critical role for Tet1 in the subsequent removal of aberrant residual and/or de novo DNA methylation (FIG. 16). This is reminiscent of the role of Tet3-driven 5mC oxidation in protection against de novo DNA methylation during zygotic DNA demethylation²⁹, suggesting that global reprogramming events require efficient protection from de novo DNA methylation following removal of 5mC to stabilise the newly acquired epigenetic state. Collectively, our study reinforces the idea that gonadal epigenetic reprogramming entails complex erasure of epigenetic information⁴ and suggests that a central function of this process is to ascertain the timely and efficient activation of GRR genes, thus enabling progression towards gametogenesis (FIG. 16).

### Methods

### Statistics and Reproducibility

All statistical tests are clearly described in the figure legends and/or in the Methods section, and exact p-values or adjusted p-values are given where possible. For WGBS data (FIGS. 3a-b, 5a, 6c-e, 7a-b, 8, 11, 12), data is derived from cells from either n=1 (E10.5 PGC sample) or n=2 (all other samples) biological replicates, with each replicate from pooled embryos (E10.5: n=39 embryos/4 litters; E11.5: n=8 embryos/1 litter; E12.5M/F: n=4 embryos/1 litter). For AbaSeq data (FIGS. 1d, 3a-b, 5c-e, 6a-f, 7a-b, 8, 11, 12, 13b), data is derived from cells from n=2 biological replicates, with each replicate from pooled embryos (E10.5: n=40 embryos/4 litters; E11.5: n=8 embryos/1 litter; E12.5M/F: n=4 embryos/1 litter). For RNA-Seq of mESCs, samples are derived from n=2 biological replicates corresponding to n=2 independently cultured samples from n=1 cell line. For PGC LC/MS, RNA-Seq and RRBS data, please see for complete details regarding the number of embryos/litters from which samples were derived. Western blots (FIGS. 13f, 15b) were performed three times with similar results, and representative blots are shown. All immunostainings (FIGS. 1e, 2a-b, FIG. 10b) were performed twice with similar results and representative images are shown. Traditional bisulphite sequencing (FIG. 10f-g) was carried out twice and a representative methylation profile is shown. For analysis of previously published WGBS (FIG. 14a-b), TAB-Seq (FIG. 5c-e), AbaSeq (FIGS. 5c-e, 6b, 14a, 14c) and ChIP-Seq (FIG. 3b, FIGS. 14a, 14c-g) datasets from mESCs (see Methods for accession numbers), other than H2Aub ChIP-Seq dataset (where n=1), biological replicates were analysed both combined (shown) and separately (not shown) to ensure reproducibility of analysis.

Mice

All animal experiments were carried out under and in accordance with a UK Home Office Project Licence in a Home-Office designated facility. Except for direct comparison with Tet1-KO PGCs, wild type PGCs were isolated from embryos produced by crossing outbred MF1 females with mixed background GOF18ΔPE-EGFP⁵ transgenic males. The sex of embryos from E12.5 onwards was determined by visual inspection of the gonads. For study of Tet1-KO PGCs, the Tet1 knockout mouse strain (B6;29S4-Tet1^{tml.IJae}/J)¹⁸ was purchased from Jackson Laboratory and bred onto the GOF18ΔPE-EGFP⁵ transgenic mouse line. Wild type and Tet1-KO PGCs were isolated from embryos produced from crosses between Tet1-heterozygous GOF18ΔPE-EGFP-homozygous females and males. For genotyping of embryos produced by crossing Tet1-heterozygous GOF18ΔPE-EGFP-homozygous males and females, PCR was always carried out twice using two different sets of primers (see below) to confirm exon 4 deletion. The sex of the embryos from E12.5 onwards was determined by visual inspection of gonads and additionally confirmed by PCR for Sry. In all cases, the mating is timed in the way that appearance of a vaginal plug at noon is defined as E0.5.

Molecular Biology

The following genotyping primers were used in this study:

(Tet1 forward primer 1)
TCAGGGAGCTCATGGAGACTA;
(Tet1 forward primer 2)
AACTGATTCCCTTCGTGCAG;
(Tet1 reverse primer)
TTAAAGCATGGGTGGGAGTC;
(Sry forward primer)
TTGTCTAGAGAGCATGGAGGGCCATGTCAA;
(Sry reverse primer)
CCACTCCTCTGTGACACTTTAGCCCTCCGA.

PGC Isolation by Flow Cytometry

PGC isolation was carried out as previously described⁴. Briefly, the embryonic trunk (E10.5) or genital ridge (E11.5-E14.5) was digested at 37° C. for 3 min using 0.05% Trypsin-EDTA (lx) (Gibco) or TrypLE Express (Thermo). Enzymatic digestion was followed by neutralization with DMEM/F-12 (Gibco) containing 15% foetal bovine serum (Gibco) and manual dissociation by pipetting. Following centrifugation, cells were re-suspended in DMEM/F-12 supplemented with hyaluronidase (300 μg/ml; Sigma), and a single cell suspension was generated by manual pipetting. Following centrifugation, cells were re-suspended in ice-cold PBS supplemented with poly-vinyl alcohol (10 μg/ml) and EGTA (0.4 mg/ml, Sigma). GFP positive cells were isolated using an Aria IIu (BD Bioscience) or Aria III (BD Bioscience) flow cytometer and sorted into ice cold PBS supplemented with poly-vinyl alcohol (10 μg/ml) and EGTA (0.4 mg/ml, Sigma).

Generation of Tet1-KO Dnmt-TKO mESCs

Tet1-KO Dnmt-TKO mESC line was generated by CRISPR/Cas9-mediated genome editing. pX330 (Addgene, #42230) with the sgRNA targeting Tet1³¹ (GGCTGCTGTCAGGGAGCTCA) was co-transfected with a reporter GFP plasmid in 5×10⁶ Dnmt-TKO mESCs²³ using Lipofectamine 3000. The day after, GFP positive cells were sorted by FACS (BD FACS Aria III) in a 96-well plate. Cells were cultured for a week before being frozen down and extracting gDNA. Colonies were screened for mutations using surveyor assay (Surveyor Mutation Detection Kit from IDT, and Taq DNA polymerase from Qiagen). Tet1-KO Dnmt-TKO mESC selected clone was further analysed by genotype sequencing, which confirmed the presence of a frameshift mutation. Loss of Tet1 was verified by RNA-Seq and western blot. The following primers were used for genotype sequencing and surveyor assay: 5′ TTGTTCTCTCCTCTGACTGC 3′ and 5′ TGATTGATCAAATAGGCCTGC 3′.

mESC Cell Culture

J1 (wild type), Dnmt-TKO²³ and Tet1-KO Dnmt-TKO mESCs were cultured in FCS/LIF medium without feeders on 0.1% gelatin. FCS/LIF medium consists of GMEM (Gibco) supplemented with 10% FCS, 0.1 mM MEM nonessential amino acids, 2 mM 1-glutamine, 1 mM sodium pyruvate, 0.1 mM 2-mercaptoethanol and mouse LIF (ESGRO, Millipore). For inhibitor experiments, mESCs were plated at a density of 1.5×10⁴/cm² and left overnight. The next morning medium was exchanged for FCS/LIF medium containing either 50 μM PRC1 inhibitor PRT4165 (Ismail et al., 2013) or DMSO control and cells pelleted at the indicated time for analysis.

AbaSeq Library Preparation

Total DNA was isolated from 10,000 sorted PGCs using the QIAamp DNA Micro Kit (Qiagen). AbaSeq libraries for 5hmC profiling were constructed as previously described¹⁵. In brief, genomic DNA was glucosylated, then digested by AbaSI enzyme (NEB). Biotinylated P1 adapters were ligated onto the AbaSI digested DNA then fragmented using a Covaris S2 sonicator (Covaris), following the manufacturer's instructions. Sheared P1-ligated DNA was then captured by mixing with Dynabeads MyOne Streptavidin C1 beads (Life Technologies) according to the manufacturer's specifications. End repair and dA-tailing were carried out on the beads by using the NEBNext End Repair Module (NEB) and the NEBNext dA-tailing Module (NEB) at 20° C. and 37° C. respectively for 30 min. P2 adapters were ligated to the random sheared ends of the dA-tailed DNA. Finally, the entire DNA was amplified using the Phusion DNA polymerase (NEB) with the addition of 300 nM forward primer (PCR_I) and 300 nM reverse primers (PCR_IIpe) for 16 cycles. The libraries were purified using AMPure XP beads (Beckman-Coulter) and sequenced on the Illumina HiSeq 2000 instrument.

Whole Genome Bisulphite Sequencing (WGBS) Library Preparation

Total DNA was isolated from 10,000 sorted PGCs using the QIAamp DNA Micro Kit (Qiagen). In some cases, unmethylated k phage DNA (Promega) was spiked in following DNA isolation to assess bisulphite conversion rate. DNA was fragmented using a Covaris S2 sonicator (Covaris), as per manufacturer's instructions. Libraries were made following the NEBNext Library Prep protocol, with methylated adaptors and the following modifications: following adaptor ligation, bisulphite conversion was carried out using the Imprint Modification Kit (Sigma); and PCR enrichment was carried out for 16 cycles using the NEXTflex Bisulphite-Seq Kit for Illumina Sequencing (Bioo Scientific) master mix and the NEBNext Library Prep universal and index primers (NEB). The libraries were purified by AMPure XP beads (Beckman-Coulter). Libraries were sequenced on the Illumina HiSeq 2000 or 2500 instrument.

Reduced Representation Bisulphite Sequencing (RRBS) Library Preparation

Total DNA from FACS-sorted PGCs isolated from individual Tet1-KO or wild type embryos was isolated using ZR-Duet DNA-RNA MiniPrep kit (Zymo), and DNA from between two to six embryos (equivalent to 1,000 to 8,000 cells) of the same genotype, stage and sex was pooled and concentrated to 26 μL final volume using the Savant SpeedVac Concentrator (Thermo) and following the manufacturer's instructions. Genomic DNA was digested by 20 units of MspI enzyme (NEB) in NEB buffer 2 at 37° C. for 3 hrs, and digested DNA was purified using AMPure XP beads (Beckman-Coulter). Libraries were made following the NEBNext Ultra DNA Library Prep protocol with methylated adaptors and the following modifications: following adaptor ligation, bisulphite conversion was carried out using the Imprint Modification Kit (Sigma); and PCR enrichment was carried out for 18 cycles using the KAPA Uracil+DNA polymerase master mix (KAPA Biosystems) and the NEBNext Library Prep universal and index primers (NEB). The libraries were purified by AMPure XP beads (Beckman-Coulter). Pooled libraries were sequenced on the Illumina HiSeq 2500 instrument, using the ‘dark sequencing’ protocol, as previously described³².

RNA-Seq Library Preparation

For study of Tet1-KO PGCs, total RNA from sorted PGCs isolated from individual Tet1-KO or wild type embryos was isolated using ZR-Duet DNA-RNA MiniPrep kit (Zymo), and RNA from between two to six embryos (equivalent to 1,000 to 8,000 cells) of the same genotype, stage and sex was pooled and concentrated to 6 μL final volume using the RNA Clean and Concentrator 5 kit (Zymo). For study of wild type PGCs isolated from embryos produced by crossing MF1 females with GOF18ΔPE-EGFP males, total RNA from 600-1,000 sorted E10.5 PGCs was isolated using the Nucleospin RNA XS kit (Macherey-Nagel). cDNA synthesis and amplification (15 cycles) was performed with the SMARTer Ultra Low Input RNA kit (Clontech) using between 100 pg and 3 ng total RNA and following the manufacturer's instructions. The amplified cDNA was fragmented by a Covaris S2 sonicator (Covaris) and following the manufacturer's instructions. Sheared cDNA was converted to sequencing libraries using the NEBNext DNA Library Prep kit (NEB), following the manufacturer's instructions and using 15 cycles of amplification. For study of mESCs, total RNA was isolated using ZR-Duet DNA-RNA MiniPrep kit (Zymo). cDNA synthesis and library prep was performed starting with 500 ng total RNA following manufacturer's instructions using the NEBNext Ultra Library Prep Kit (NEB) and the NEBNext Poly(A) mRNA Magnetic Isolation Module (NEB). All libraries were purified by AMPure XP beads (Beckman-Coulter) and sequenced on the Illumina HiSeq 2500 instrument.

Bioinformatics

Whole Genome Bisulphite Sequencing (WGBS) and Tet-Assisted Bisulphite Sequencing (TAB-Seq) Alignment and Downstream Analysis

Raw reads were first trimmed using Trim Galore (version 0.3.1) with the --paired --trim1 options. Alignments were carried out to the mouse genome (mm9, NCBI build 37) with Bismark (version 0.13.0) with the -n 1 parameter; where appropriate, the λ phage genome was added as an extra chromosome. Aligned reads were deduplicated with deduplicate_bismark. Where appropriate, the bisulphite conversion rate was computed using reads aligned to the λ phage genome and using the to-mr script (parameters: -m bismark) and bsrate script (paramters: -N) of Methpipe (version 3.3.1). CpG methylation calls were extracted from the deduplicated mapping output using the Bismark methylation extractor.

The number of methylated and unmethylated cytosines in a CpG context was extracted using bismark2bedGraph and coverage2cytosine. Symmetric CpGs were merged with custom R script. For all downstream analysis, only symmetric CpGs with minimum 8× coverage were used. All WGBS analysis was carried out on data from merged biological replicates. For assessing DNA modification levels at specific repetitive elements, Bismark (version 0.14.4) was used to map all reads from each data set against consensus sequences constructed from Repbase with the −n 1 parameter set. CpG methylation calls were extracted from the mapping output using the Bismark methylation extractor (version 0.14.4).

The mapBed function of BEDtools (version 2.24.0) was used to compute the combined 5mC/5hmC level for the following genomic features: 1) all 2 kb windows (containing a minimum 4 symmetric CpGs); 2) gene promoters (defined as Ensembl 67 gene start sites −1 kB/+500 bp); 3) gene bodies (defined as the region contained within Ensembl 67 gene start and gene end sites); 4) putative active enhancers in day 6 PGCLCs³³; 5) imprint control regions; 6) CpG islands (UCSC); 7) intergenic regions. For metagene plots, a genomic feature was divided into equally sized bins using BEDtools (version 2.24.0), including: 1) gene bodies (defined as the region contained within Ensembl 67 gene start and gene end sites)+/−0.5*gene body length (100 bins); 2) putative active enhancers in day 6 PGCLCs 33 +/−1*putative active enhancer length (90 bins); and 3) CpG islands (UCSC)+/−1*CpG island length (90 bins). In all cases, the combined 5mC/5hmC level was expressed as the mean of individual CpG sites.

For k-means clustering of the combined mean 5mC/5hmC levels, high CpG (HCP), intermediate CpG (ICP) and low CpG (LCP) promoters, as defined using the same parameters as previously published¹⁹³⁴. Briefly, LCPs contain no 500-bp window with a CpG ratio >0.45; HCPs contain at least one 500-bp window with a CpG ratio >0.65 and GC content >55%; ICPs do not meet the previous criteria.

For determining locus-specific methylation levels in wild type mESCs grown in serum-containing media, raw WGBS reads were downloaded from GSE48519³⁰ and processed as above. TAB-Seq reads for E14 mESCs were downloaded from GSE36173³⁵ and processed as above, with the exception that only symmetric CpGs with minimum 12× coverage were used.

AbaSeq Alignment and Downstream Analysis

For the uniquely mappable part of the genome, AbaSeq reads were processed as previously described¹⁵. In brief, raw sequencing reads were trimmed for adaptor sequences and low quality bases using Trim Galore. The trimmed reads were mapped to the mouse genome (mm9, NCBI build 37) using Bowtie (version 0.12.8) with parameters −n 1 -l25 --best --strata -m 1. Calling of 5hmC was based on the recognition sequence and cleavage pattern of the AbaSI enzyme (5′-C₁₁₋₁₃↓N₉₋₁₀G-3′/3′-GN₉₋₁₀↓N₁₁₋₁₃C-5′) using custom Perl scripts. For assessing relative enrichment of 5hmC at repetitive elements and non-repetitive elements, AbaSeq alignments were divided into two groups: unique (single best alignment) and ambiguous (map to multiple locations with equal alignment score). Both groups were then mapped to the repetitive elements defined by the RepeatMasker track of mm9 (UCSC Genome Browser) separately. For comparison with 5hmC levels in mESCs, AbaSeq reads were downloaded from GSE42898¹⁵ and aligned in the same way.

For quantification of relative 5hmC levels at symmetric CpGs in the uniquely mapped part of the genome, the number of counts per symmetric CpG for a given sample were normalised to the combined number of uniquely mapped and ambiguously mapped reads for a given library, and then further multiplied by a stage-specific normalisation factor based on the mean 5hmC level for each stage as computed by LC/MS (E14 ESC=1.64; E10.5=1.0; E11.5=1.13; E12.5F=0.76; E12.5M=1.0). All symmetric CpGs falling within genomic intervals blacklisted by the mouse (mm9) ENCODE project were excluded from all further downstream analysis. Unless stated otherwise, all AbaSeq analysis was carried out on data from merged biological replicates.

The mapBed function of BEDtools (version 2.24.0) was used to compute the 5hmC level for the same genomic features as was carried out with WGBS datasets (see above). In all cases, the 5hmC level was expressed as the mean of individual CpG sites.

To identify 5hmC enriched or depleted regions in E10.5 and E11.5 PGCs, the mm9 genome was first divided into 2 kb windows (minimum 4 symmetric CpGs) and the mean 5hmC level for each window was computed using BEDtools (version 2.24.0). To determine the significance of 5hmC enrichment in each 2 kB window, upper-tail (to determine 5hmC enriched regions) or lower-tail (to determine 5hmC depleted regions) Poisson probability p-values were computed using ppois(x, λ), where x is the observed 5hmC mean value for each 2 kb window and λ is the mean of 5hmC mean values for all 2 kb windows at E10.5. Benjamini-Hochberg correction was then applied to correct for multiple testing, giving a final adjusted upper-tail and lower-tail p-value for each 2 kb window. Windows with adjusted upper-tail p-value<0.05 were considered relatively enriched for 5hmC while windows with adjusted lower-tail p-value<0.05 were considered relatively depleted for 5hmC.

For assessing relative enrichment of 5hmC at specific repetitive elements, Bowtie was used to map all reads from each data set against consensus sequences constructed from Repbase with parameters −n 1−M 1 --strata -best. The number of reads mapped to each sequence within a given sample was first normalised to the library size of that particular sample, and then normalised to both a stage-specific normalisation factor based on the mean 5hmC level for each stage as computed by LC/MS (E10.5=1.0; E11.5=1.13; E12.5F=0.76; E12.5M=1.0) and the mean proportion of reads mapped to a given sequence in E10.5 PGCs.

Reduced Representation Bisulphite Sequencing (RRBS) Alignment and Downstream Analysis

Raw RRBS reads were first trimmed using Trim Galore (version 0.3.1) with --rrbs parameter. Alignments were carried out to the mouse genome (mm9, NCBI build 37) with Bismark (version 0.13.0) with the -n 1 parameter. CpG methylation calls were extracted from the mapping output using the Bismark methylation extractor (version 0.13.0). The number of methylated and unmethylated cytosines in a CpG context was extracted using bismark2bedGraph.

RnBeads (version 1.0.0) and RnBeads.mm9 (version 0.99.0) were used to identify differentially methylated regions between two test groups for the following genomic features, with filtering.missing.value.quantile set to 0.95 and filtering.missing.coverage.threshold set to 8: 1) all 2 kb windows (containing a minimum 4 symmetric CpGs); 2) gene promoters (defined as Ensembl 67 gene start sites −1 kB/+500 bp); and 3) imprint control regions (mm9 genome). The following was extracted from the output of RnBeads: 1) the mean methylation level for each group (i.e. stage, sex and/or genotype) for each commonly covered test region; 2) the difference in methylation means between two groups for each commonly covered test region; and 3) the p-value representing the significance of the difference in methylation means between two groups for each commonly covered test region. Differentially methylated regions were identified as regions with a p-value<0.05 and a difference in methylation means between two groups greater than 10%.

For assessing DNA modification levels at specific repetitive elements, Bismark (version 0.14.4) was used to map all reads from each data set against consensus sequences constructed from Repbase with the -n 1 parameter set. CpG methylation calls were extracted from the mapping output using the Bismark methylation extractor (version 0.14.4). The number of methylated and unmethylated cytosines in a CpG context were extract using bismark2bedGraph and coverage2cytosine. Differentially methylated consensus repeats were identified as regions with a p-value<0.05 (as computed by two-sided Student's t-test) and a difference in methylation means between two groups greater than 10%.

hMeDIP Alignment and Downstream Analysis

Raw hMeDIP-Seq and input reads for E14 mESCs were downloaded from GSE28500³⁶ and aligned to the mouse genome (mm9, NCBI build 37) with Bowtie (version 0.12.8) with parameters -n 2 -l25 -m 1. BEDtools multicov was used to identify the number of hMeDIP and input reads overlapping each 2 kB window (containing a minimum 4 symmetric CpGs). Final 5hmC levels for each 2 kB window were determined by first normalising the number of overlapping hMeDIP reads (normalised to library size) by the number of overlapping input reads (normalised to library size) and then by dividing this value by the number of symmetric CpGs contained within the 2 kB window.

ChIP-Seq Alignment and Downstream Analysis

For putative active enhancer calling, raw ChIP-Seq reads for H3K4me3, H3K27me3 and H3K27Ac in day 6 PGC-like cells (PGCLCs) were downloaded from GSE60204³³ and raw ChIP-Seq reads for H3K4me3, H3K27me3, H3K4mel and H3K27Ac in wild type mESCs were downloaded from GSE48519³⁰. Reads were aligned to the mouse genome (mm9, NCBI build 37) with Bowtie (version 0.12.8 or version 1.0.0) with parameters -n 2 -l 25 -m l and -C where appropriate. Subsequent ChIP-Seq analysis was carried out on data from merged biological replicates. To identify putative active enhancers, the inventors first generated an 8-state chromatin model using ChromHMM. Putative active enhancers were defined as all regions not overlapping any potential promoter regions (Ensembl 67 gene start sites −1 kB/+500 bp) and contained within the (H3K27Ac⁺/H3K4me3⁻/H3K27me3⁻) chromatin state in day 6 PGCLCs or (H3K4me⁺/H3K27Ac⁺/H3K4me3⁻/H3K27me3⁻) in wild type mESCs.

For analysis of epigenetic modifications and modifiers around transcription start sites (Ensembl 67): raw ChIP-Seq reads for: Tet1 binding in wild type serum-grown mESCs was downloaded from GSE24843²¹; H2AK19Ub1 levels in wild type serum-grown mESCs were downloaded from GSE34520³⁷; Ring1b binding in wild type serum-grown mESCs were downloaded from ERP005575³⁸; and for H3K4me3 in wild type and Tet1-KO serum-grown mESCs were downloaded from GSE48519³⁰. Reads were aligned to the mouse genome (mm9, NCBI build 37) with Bowtie (version 0.12.8 or version 1.0.0) with parameters -n 2 -l 25 -m 1. Subsequent ChIP-Seq analysis was carried out on data from merged biological replicates. For computing ChIP-Seq signal around transcription start sites (TSS), the genomic interval around the Ensembl 67 gene start sites+/−5 kB (or 2 kB) was divided into 100 (or 40) equally sized bins using BED tools make windows. BED tools multicov was then used to compute the number of test and control reads overlapping each bin. The total number of test and control reads per bin for each sample were normalised to the appropriate library size, and fold enrichment for each bin was determined by dividing the number of normalised ChIP-Seq test sample reads by the number of normalised ChIP-Seq control sample reads. For computing ChIP-Seq signal at gene promoters, the genomic interval around the Ensembl 67 gene start sites +500 bp/−1 kB was

RNA-Seq Alignment and Downstream Analysis

For study of Tet1-KO and Tet1-WT PGCs, Illumina and Smart-seq adapters from the sequencing reads were first trimmed using Trimmomatic. For other RNA-Seq libraries, fastq files generated from output of next generation sequencing were used directly for alignment. RNA-Seq reads were aligned to the mouse genome (mm9, NCBI build 37) with Bowtie (version 0.12.8) and Tophat (version 2.0.2) with options -N 2 --b2-very-sensitive --b2-L 25. Annotations from Ensembl Gene version 67 were used as gene model with Tophat. Read counts per annotated gene were computed using HTSeq (version 0.5.3p9) and expression level of each gene was quantified by computing the number of fragments detected per kilobase per million of reads (FPKM) using custom R script. Genes were assigned to an expression level bin based on the mean FPKM values of the two biological replicates. Differential expression analysis was performed using DESeq2 (version 1.6.3), and genes with an adjusted p-value<0.05 were considered differentially expressed. For determining gene expression levels in wild type and Dnmt1-conditional knockout and matched wild type E10.5 PGCs, raw RNA-Seq reads were downloaded from GSE74938²⁴ and processed as above.

HCPs methylated and demethylating in PGCs during epigenetic reprogramming (cluster 3, FIG. 4A) were ranked based on the significance of activation (α) between gene expression in E10.5 and E14.5 PGCs (FIG. 4B). In the case where β represents the directionality of fold change (i.e. if log 2(FC)<0, β=−1; else β=+1) and γ represents the adjusted p-value as computed by DESeq2, α=β×(1−γ). For comparing expression levels of the GRR gene set in 1) wild type, Dnmt-TKO, and Tet1-KO Dnmt-TKO mESCs (FIG. 6A); 2) in wild type+6h DMSO treatment, Dnmt-TKO+6h DMSO treatment, wild type+6h PRT4165 treatment, Dnmt-TKO+6h PRT4165 treatment (FIG. 6C); 3) Tet1-KO E14.5 PGCs against wild type E14.5 PGCs (FIG. 5B); or 3) Dnmt1-CKO E10.5 PGCs against wild type E10.5 PGCs (FIG. 13G) pairwise differential expression analysis was initially carried out by DESeq2 for each condition against each other condition. For each pairwise differential expression test, each gene was assigned a statistic α, where if β represents the log₂(FC) and γ represents the adjusted p-value as computed by DESeq2, α=β×(1−γ). The ranked gene list based on a was subsequently used for gene set enrichment analysis (GSEA) for testing general up or down-regulation of the combined GRR gene sets and GSEA hallmark gene sets, and GSEA FWER-adjusted p-values were subsequently used. For overlap between germline reprogramming responsive genes and genes repressed by PRC1 in PGCs (FIG. 6B), the list of genes called up-regulated in E11.5 and/or E12.5 PRC1-KO PGCs was downloaded from ²⁶.

For classification of GRR genes (FIG. 14, Table 1), pairwise differential expression analysis was first carried out. 5mC-reprogramming dependent GRR genes were defined as genes: 1) up-regulated in Dnmt-TKO vs WT, Dnmt-TKO+PRC1 inhibitor vs WT, and Dnmt-TKO+PRC1 inhibitor vs WT+PRC1 inhibitor; and 2) not up-regulated in WT+PRC1 inhibitor vs WT. PRC1-reprogramming dependent GRR genes were defined as genes: 1) up-regulated in WT+PRC1 inhibitor vs WT, Dnmt-TKO+PRC1 inhibitor vs WT, and Dnmt-TKO+PRC1 inhibitor vs Dnmt-TKO; and 2) not up-regulated in Dnmt-TKO vs WT. 5mC/PRC1-reprogramming dependent GRR genes were defined as genes either: 1) up-regulated in WT+PRC1 inhibitor vs WT, Dnmt-TKO vs WT, Dnmt-TKO+PRC1 inhibitor vs WT, Dnmt-TKO+PRC1 inhibitor vs Dnmt-TKO, and Dnmt-TKO+PRC1 inhibitor vs WT+PRC1 inhibitor; or 2) up-regulated in Dnmt-TKO+PRC1 inhibitor vs WT, Dnmt-TKO+PRC1 inhibitor vs Dnmt-TKO, and Dnmt-TKO+PRC1 inhibitor vs WT+PRC1 inhibitor, and not up-regulated in WT+PRC1 inhibitor vs WT and Dnmt-TKO vs WT. 5mC/PRC1 reprogramming independent or insufficient GRR genes were defined as genes not-upregulated in Dnmt-TKO vs WT, Dnmt-TKO+PRC1 inhibitor vs WT, and Dnmt-TKO+PRC1 inhibitor vs WT+PRC1 inhibitor, and WT+PRC1 inhibitor vs WT. Genes that do fall into one of these five classes were described as low confidence classification (l.c.c.) genes.

Tet1 and 5mC/5hmC Detection by Immunofluorescence

The embryonic trunk (E10.5) or genital ridge (E12.5/E13.5) was first fixed in 2% PFA (in PBS) for 30 min at 4° C. Following fixation, tissue was washed in PBS three times for 10 min and then incubated in 15% sucrose in PBS overnight. After rinsing with 1% BSA in PBS the following day, the tissue was embedded in OCT Embedding Matrix (Thermo Scientific Raymond Lamb) and frozen using liquid nitrogen. Samples were subsequently stored at −80° C. A Leica CM 1950 cryostat was used to cut 10 μm sections from the frozen embedded tissue. Sections were settled on poly-lysine slides (Thermo Scientific) and post-fixed with 2% PFA in PBS for 3 minutes.

For detection of Tet1, sections were washed three times for 5 min with PBS. After incubating for 30 min at room temperature in 1% BSA/PBS containing 0.1% Triton X-100, the sections were incubated with primary antibodies listed at 4° C. overnight in the same buffer. Sections were subsequently washed three times in 1% BSA/PBS containing 0.1% Triton X-100 for 5 min and incubated with secondary antibodies in the same buffer for 1 hour in the dark at room temperature. Secondary antibody incubation was followed by three 5 min washes with PBS. DNA was then stained with DAPI (100 ng/ml). After a final wash in PBS for 10 min, the sections were mounted with Vectashield (Vector Laboratories).

For detection of 5hmC/5mC, sections were washed three times for 5 min with PBS. Post-fixed sections were first permeabilized for 30 min with 0.5% Triton X-100 (in 1% BSA/PBS), and subsequently treated with RNase A (10 mg/ml; Roche) in 1% BSA/PBS for 1 hour at 37° C. Following three 5 min washes with PBS, sections were incubated with 4N HCl for 10-20 min at 37° C. to denature genomic DNA, followed by three 10 min washes with PBS. After incubating for 30 min at room temperature in 1% BSA/PBS containing 0.1% Triton X-100, the sections were incubated with primary antibodies listed at 4° C. overnight in the same buffer. Sections were subsequently washed three times in 1% BSA/PBS containing 0.1% Triton X-100 for 5 min and incubated secondary antibodies in the same buffer for 1 hour in the dark at room temperature. Secondary antibody incubation was followed by three 5 min washes with PBS. DNA was then stained with propidium iodide (PI) (0.25 mg/ml). After a final wash in PBS for 10 min, the sections were mounted with Vectashield (Vector Laboratories).

The following primary antibodies were used in this study: anti-SSEA1 (gifted by Dr P. Beverly via Dr G. Durcova Hills); anti-MVH (Abcam 27591 or Abcam 13840); anti-5hmC (Active motif 39791), anti-5mC (Diagenode C15200081-100); anti-Tet1 (GeneTex GTX125888); anti-GFP (Abcam 5450). The following secondary antibodies were used in this study: Alexa Fluor 647 Goat anti-Mouse IgM (Invitrogen A21238); Alexa Fluor 488 Goat anti-Rabbit IgG (Invitrogen A11008); Alexa Fluor 405 Goat anti-Mouse IgG 1:300 (Invitrogen A31553); Alexa Fluor 488 Goat anti-Mouse IgG 1:300 (Invitrogen A11001); Alexa Fluor 405 Goat anti-Rabbit IgG 1:300 (Invitrogen A31556); Alexa Fluor 568 Donkey anti-Rabbit IgG (Invitrogen A10042); Alexa Fluor 488 Donkey anti-Goat IgG (Invitrogen A11055).

Locus-Specific Bisulphite Sequencing

Bisulphite treatment of genomic DNA was carried out using the Imprint DNA modification kit (Sigma). The following primers were used for the semi-nested amplification of the Dazl promoter: F1: GATTTTTGTTATTTTTTAGTTTTTTTAGGAT; F2: TTTATTTAAGTTATTATTTTAAAAATGGTATT; R: AGAAACAAGCTAGGCCAGCTGAGAGAATTCT. The following primers were used for the semi-nested amplification of the IG-DMR ICR: F1: GTGTTAAGGTATATTATGTTAGTGTTAGG; F2: ATATTATGTTAGTGTTAGGAAGGATTGTG; R: TACAACCCTTCCCTCACTCCAAAAATT. The following primers were used for the nested amplification of the Peg3 ICR: F1: TTTTTAGATTTTGTTTGGGGGTTTTTAATA; F2: TTGATAATAGTAGTTTGATTGGTAGGGTGT; R1: AATCCCTATCACCTAAATAACATCCCTACA; R2: ATCTACAACCTTATCAATTACCCTTAAAAA. Methylation levels were assessed by QUMA, using default settings with duplicate bisulphite sequences excluded.

Mass Spectrometry

Genomic DNA from between 100 and 2,000 FACS-sorted PGCs was extracted using ZR-Duet DNA/RNA Miniprep kit (Zymo Reasearch) following manufacturer instructions and eluted in LC/MS grade water. DNA was digested to nucleosides using a digestion enzyme mix provided by NEB. A dilution-series made with known amounts of synthetic nucleosides and the digested DNA were spiked with a similar amount of isotope-labelled nucleosides (provided by Dr T. Carell (LMU, Germany)) and separated on an Agilent RRHD Eclipse Plus C18 2.1×100 mm 1.8u column by using the UHPLC 1290 system (Agilent) and an Agilent 6490 triple quadrupole mass spectrometer. To calculate the quantity of individual nucleosides, standard curves representing the ratio of unlabelled over isotope-labelled nucleosides were generated and used to convert the peak-area values to corresponding quantity. Threshold for quantification is a signal-to-noise (calculated with a peak-to-peak method) above 10.

Western Blot

mESCs were lysed by sonication in RIPA buffer (150 mM sodium chloride, 1.0% Triton X-100, 0.5% sodium deoxychlorate, 0.1% sodium dodecylsulfate, 50 mM Tris pH 8.0) and protease-inhibitor cocktail (Roche, 11 697 498 001). Cell debris were removed by centrifugation at 14000 g 5 min 4° C. Protein was quantified using the BCA protein assay (Thermo, 23227). 2 μg (for H2A and H2Aub) or 20 μg (for Tet1) of each protein extract was loaded onto an 15% or 8% SDS polyacrylamide gel and transferred to a PVDF membrane after electrophoresis. Membranes were blocked with 5% BSA for 1 hour and then incubated overnight at 4° C. with primary antibodies at the following dilutions: anti-H2A antibody (Abcam, 18255) 1:2000; anti-ubiquityl H2A antibody (Cell Signalling 8240) 1:2000; anti-Tet1 antibody [N1] (GeneTex GTX125888) 1:1000; anti-Lamin B antibody (C20) (Santa Cruz Biotechnologies, sc-6216) 1:10000. Donkey anti-rabbit IgG-HRP (Santa Cruz Biotechnologies, sc-2077) or donkey anti-goat IgG-HRP (Santa Cruz Biotechnologies, sc-2056) secondary antibody were incubated for 1h at room temperature. Blots were developed by using Luminata Crescendo Western HRP substrate (EMD Milipore).

### REFERENCES

Alphabetised References:
Chung et al., 2008 Human Embryonic Stem Cell Lines Generated without Embryo Destruction. Cell Stem Cell. 2(2) 113-117. Epub 2008 Jan 10
Galdon et al. In Vitro Spermatogenesis: How Far from Clinical Application? Curr Urol Rep 17; 49 (2016)
Hill et al. Epigenetic reprogramming enables the primordial germ cell-to-gonocyte transition, Nature; 555(7696): 392-396; 15 Mar. 2018.
Jiang et al., Pluripotency of mesenchymal stem cells derived from adult marrow. Nature 418, 41-49, 2002
Kaji et al Virus-free induction of pluripotency and subsequent excision of reprogramming factors. Nature. Online publication 1 Mar. 2009
Kanatsu-Shinohara et al., Generation of pluripotent stem cells from neonatal mouse testis. Cell 119, 1001-1012,2004
Phillips et al. Spermatogonial stem cell regulation and spermatogenesis. Phil Trans R Soc B 365, 1663-1678 (2010)
Matsui et al., Derivation of pluripotential embryonic stem cells from murine primordial germ cells in culture. Cell 70, 841-847, 1992
Medrano et al. Human somatic cells subjected to genetic induction with six germ line-related factors display meiotic germ cell-like features. Scientific Reports 6:24956: 10.1038 (2016)
Numbered References (MAIN):
1 Lesch, B. & Page, D. Genetics of germ cell development. Nat Rev Genet 13, 781-794 (2012).
2 Guibert, S., Forn6, T. & Weber, M. Global profiling of DNA methylation erasure in mouse primordial germ cells. Genome Res 22, 633-641 (2012).
3 Hackett, J. et al. Germline DNA demethylation dynamics and imprint erasure through 5-hydroxymethylcytosine. Science 339, 448-452 (2013).
4 Hajkova, P. et al. Chromatin dynamics during epigenetic reprogramming in the mouse germ line. Nature 452, 877-881 (2008).
Hajkova, P. et al. Epigenetic reprogramming in mouse primordial germ cells. Mech Dev 117, 15-23 (2002).
6 Hill, P., Amouroux, R. & Hajkova, P. DNA demethylation, Tet proteins and 5-hydroxymethylcytosine in epigenetic reprogramming: an emerging complex story. Genomics 104, 324-333 (2014).
7 Lee, J. et al. Erasing genomic imprinting memory in mouse clone embryos produced from day 11.5 primordial germ cells. Development 129, 1807-1817 (2002).
8 Seisenberger, S. et al. The dynamics of genome-wide DNA methylation reprogramming in mouse primordial germ cells. Mol Cell 48, 849-862 (2012).
9 Yamaguchi, S. et al. Tet1 controls meiosis by regulating meiotic gene expression. Nature 492, 443-447 (2012).
Yamaguchi, S., Shen, L., Liu, Y., Sendler, D. & Zhang, Y. Role of Tet1 in erasure of genomic imprinting. Nature 504, 460-464 (2013).
11 Hajkova, P. et al. Genome-wide reprogramming in the mouse germ line entails the base excision repair pathway. Science 329, 78-82 (2010).
12 Hayashi, K. et al. Offspring from oocytes derived from in vitro primordial germ cell-like cells in mice. Science 338, 971-975 (2012).
13 Hayashi, K., Ohta, H., Kurimoto, K., Aramaki, S. & Saitou, M. Reconstitution of the mouse germ cell specification pathway in culture by pluripotent stem cells. Cell 146, 519-532 (2011).
14 Hikabe, O. et al. Reconstitution in vitro of the entire cycle of the mouse female germ line. Nature 539, 299-303 (2016).
Sun, Z. et al. High-resolution enzymatic mapping of genomic 5-hydroxymethylcytosine in mouse embryonic stem cells. Cell Rep 3, 567-576 (2013).
16 Huang, Y. et al. The behaviour of 5-hydroxymethylcytosine in bisulfite sequencing. PLoS One 5, e8888 (2010).
17 Yamaguchi, S. et al. Dynamics of 5-methylcytosine and 5-hydroxymethylcytosine during germ cell reprogramming. Cell Res 23, 329-339 (2013).
18 Dawlaty, M. et al. Tet1 is dispensable for maintaining pluripotency and its loss is compatible with embryonic and postnatal development. Cell Stem Cell 9, 166-175 (2011).
19 Weber, M. et al. Distribution, silencing potential and evolutionary impact of promoter DNA methylation in the human genome. Nat Genet 39, 457-466 (2007).
Tahiliani, M. et al. Conversion of 5-methylcytosine to 5-hydroxymethylcytosine in mammalian DNA by MLL partner TET1. Science 324, 930-935 (2009).
21 Williams, K. et al. TET1 and hydroxymethylcytosine in transcription and DNA methylation fidelity. Nature 473, 343-348 (2011).
22 Vella, P. et al. Tet proteins connect the O-linked N-acetylglucosamine transferase Ogt to chromatin in embryonic stem cells. Mol Cell 49, 645-656 (2013).
23 Tsumura, A. et al. Maintenance of self-renewal ability of mouse embryonic stem cells in the absence of DNA methyltransferases Dnmt1, Dnmt3a and Dnmt3b. Genes Cells 11, 805-814 (2006).
24 Hargan-Calvopina, J. et al. Stage-Specific Demethylation in Primordial Germ Cells Safeguards against Precocious Differentiation. Dev Cell 39, 75-86 (2016).
Mansour, A. et al. The H3K27 demethylase Utx regulates somatic and germ cell epigenetic reprogramming. Nature 488, 409-413 (2012).
26 Yokobayashi, S. et al. PRC1 coordinates timing of sexual differentiation of female primordial germ cells. Nature 495, 236-240 (2013).
27 Ismail, I., McDonald, D., Strickfaden, H., Xu, Z. & Hendzel, M. A small molecule inhibitor of polycomb repressive complex 1 inhibits ubiquitin signaling at DNA double-strand breaks. J Biol Chem 288, 26944-26954 (2013).
28 Deplus, R. et al. TET2 and TET3 regulate GlcNAcylation and H3K4 methylation through OGT and SET1/COMPASS. EMBO J32, 645-655 (2013).
29 Amouroux, R. et al. De novo DNA methylation drives 5hmC accumulation in mouse zygotes. Nat Cell Biol 18, 225-233, doi:10.1038/ncb3296 (2016).
30 Hon, G. et al. 5mC Oxidation by Tet2 Modulates Enhancer Activity and Timing of Transcriptome Reprogramming during Differentiation. Mol Cell 56, 286-297 (2014).
31 Yang, H. et al. One-step generation of mice carrying reporter and conditional alleles by CRISPR/Cas-mediated genome engineering. Cell 154, 1370-1379 (2013).
32 Boyle, P. et al. Gel-free multiplexed reduced representation bisulfite sequencing for large-scale DNA methylation profiling. Genome Biol 13, R92 (2012).
33 Kurimoto, K. et al. Quantitative Dynamics of Chromatin Remodeling during Germ Cell Specification from Mouse Embryonic Stem Cells. Cell Stem Cell 16, 517-532 (2015).
34 Borgel, J. et al. Targets and dynamics of promoter DNA methylation during early mouse development. Nat Genet 42, 1093-1100 (2010).
35 Yu, M. et al. Base-resolution analysis of 5-hydroxymethylcytosine in the mammalian genome. Cell 149, 1368-1380 (2012).
36 Xu, Y. et al. Genome-wide regulation of 5hmC, 5mC, and gene expression by Tet1 hydroxylase in mouse embryonic stem cells. Mol Cell 42, 451-464 (2011).
37 Brookes, E. et al. Polycomb associates genome-wide with a specific RNA polymerase II variant, and regulates metabolic genes in ESCs. Cell Stem Cell 10, 157-170 (2012).
38 Cooper, S. et al. Targeting polycomb to pericentric heterochromatin in embryonic stem cells reveals a role for H2AK119ul in PRC2 recruitment. Cell Rep 7, 1456-1470 (2014).
39 Hashimoto, H. et al. Structure of a Naegleria Tet-like dioxygenase in complex with 5-methylcytosine DNA. Nature 506, 391-395 (2014).

All references referred to above are hereby incorporated by reference.

1. An in vitro method of producing a meiotically competent cell, the method comprising:
(i) providing a precursor cell,
(ii) inhibiting methylation of the genomic DNA of the precursor cell,
(iii) treating the precursor cell with an inhibitor of a polycomb repressive complex, and then
(iv) propagating the precursor cell for a period of time and under culture conditions suitable for the precursor cell to become a meiotically competent cell;
wherein step (ii) and step (iii) may be performed simultaneously or sequentially in either order.

2. The method according to claim 1, wherein the precursor cell is derived from a sample that has been obtained from a subject.

3. The method according to claim 1, wherein the precursor cell is a stem cell or a primordial germ cell-like cell (PGCLC).

4. The method according to claim 3, wherein the stem cell is an iPS cell.

5. The method according to claim 1, wherein said precursor cell expresses Tet1, or begins expressing Tet1 following step (i) and/or (ii).

6. The method according to claim 1, wherein said inhibiting step (ii) and said treating step (iii) are sufficient to induce expression of germline reprogramming responsive (GRR) genes by the precursor cell during propagating step (iv).

7. The method according to claim 6, wherein the expression of the GRR genes is associated with or induced by recruitment of a transcriptional activator.

8. The method according to claim 7, wherein the transcriptional activator is Tet1.

9. The method according to claim 5, wherein Tet1 expression is exogenously provided or enhanced.

10. The method according to claim 1, wherein Tet1 protein is exogenously introduced into the precursor cell before or during step (iv).

11. The method according to claim 9, wherein the exogenously provided or exogenously introduced Tet1 is a Tet1 fusion construct that is targeted to one or more specific genomic regions.

12. The method according to claim 1, further comprising:
(v) detecting the expression level of one or more GRR genes in the cell.

13. The method according to claim 12, wherein step (v) is performed on the meiotically competent cell following step (iv).

14. The method according to claim 1, wherein the inhibitor of polycomb repressive complex is a PRC1 inhibitor and/or a PRC2 inhibitor.

15. (canceled)

16. The method according to claim 14, wherein the PRC1 inhibitor is PRT4165.

17. The method according to claim 1, wherein the inhibitor of polycomb repressive complex is an RNAi molecule.

18. The method according to claim 1, wherein step (ii) is performed by treating the precursor cell with an agent that reduces genomic DNA methylation.

19. The method according to claim 18, wherein the agent that reduces genomic DNA methylation is a DNA methyltransferase inhibitor, an agent that prevents the deposition of DNA methylation, or an agent that inhibits the maintenance of DNA methylation.

20. The method according to claim 19, wherein the agent that reduces genomic DNA methylation is a DNA methyltransferase inhibitor, optionally wherein the DNA methyltransferase inhibitor is a DNMT1 inhibitor.

21. (canceled)

22. The method according to claim 20, wherein the DNA methyltransferase inhibitor is SGI 1027, 5-azacytidine, or an RNAi molecule.

23. (canceled)

24. The method according to claim 1, wherein step (ii) is performed by using gene-editing to inactivate a DNA methyltransferase gene or a component of DNA methylation machinery.

25. A meiotically competent cell produced by the method of any one of the preceding claims.

26. A method of inducing gametogenesis, the method comprising treating the meiotically competent cell according to claim 25 with retinoic acid.

27. (canceled)

28. (canceled)

29. A gametocyte produced by the method according to claim 21, or a gamete derived therefrom.

30. (canceled)

31. A kit for the in vitro production of the meiotically competent cell according to claim 25, the kit comprising a methylation inhibitor, and an inhibitor of a poly comb repressive complex.

32. (canceled)

33. A method of assessing the fertility of a mammal, the method comprising determining the nucleic acid sequence and/or epigenetic status of one or more germline reprogramming responsive (GRR) genes in a cell that has been obtained from the mammal.

34. A method of determining the meiotic competency of a cell, the method comprising determining the nucleic acid sequence and/or epigenetic status and/or gene expression level of one or more germline reprogramming responsive (GRR) genes in the genomic DNA of the cell.
